(19) United States
(12) Patent Application Publication
(10) Pub. No.: US 2017/0049816 A1
(43) Pub. Date: Feb. 23, 2017

(54) Methods of Benchmarking Wellness with a Stable Reactive Oxygen Species Solution

(71) Applicant: Reoxcyn Discoveries Group, Inc., Salt Lake City, UT (US)

(72) Inventor: Bristol Sorensen, San Leandro, CA (US)

(21) Appl. No.: 14/231,127

(22) Filed: Mar. 31, 2014

Related U.S. Application Data
(60) Provisional application No. 61/813,451, filed on Apr. 18, 2013.

(51) Int. Cl. A61K 33/40; C02F 1/461; G01N 33/487

(52) U.S. Cl. CPC A61K 33/40; G01N 33/48707; C02F 1/461; C02F 2303/04

(57) ABSTRACT

Methods of benchmarking wellness are described herein.

### TECHNICAL FIELD

Methods of benchmarking wellness are described herein.

### BACKGROUND

Redox signaling deals with the action of a set of several simple reactive signaling molecules that are mostly produced by the mitochondria residing inside human cells during the metabolism of sugars. These reactive signaling molecules are categorized into two general groups, Reactive Oxygen Species (ROS), which comprise oxidants, and Reduced Species (RS), which comprise reductants. These fundamental universal signaling molecules in the body are the simple but extremely important reactive signaling molecules that are formed from combinations of the atoms (Na, Cl, H, O, N) that are readily found in the saline bath that fills the inside of the cells (cytosol). All of the molecular mechanisms inside healthy cells float around in this saline bath and are surrounded by a balanced mixture of such reactive signaling molecules. A few examples of the more than 20 reactive molecules formed from these atoms inside the cell, some of which are discussed herein, are superoxide, hydrogen peroxide, hypochlorous acid and nitric oxide.

Such reactive signaling molecules are chemically broken down by specialized enzymes placed at strategic locations inside the cell. Some of these protective enzymes are classified as antioxidants such as Glutathione Peroxidase and Superoxide Dismutase. In a healthy cell, the mixtures of these reactive signaling molecules are broken down by the antioxidant enzymes at the same rate that they are produced by the mitochondria. As long as this homeostatic balance is maintained, the cell's chemistry is in balance and all is well.

When damage occurs to the cell, for any number of reasons, including bacterial or viral invasion, DNA damage, physical damage or toxins, this homeostatic balance is disturbed and a build-up of oxidants or reductants occurs in the cell. This condition is known as oxidative stress and it acts as a clear signal to the cell that something is wrong. The cell reacts to this signal by producing the enzymes and repair molecules necessary to attempt repairs to the damage and it also can send messengers to activate the immune system to identify and eliminate threats. If oxidative stress persists in the cell for more than a few hours, then the cell's repair attempts are considered unsuccessful and the cell kills and dismantles itself and is replaced by the natural cellular division of healthy neighboring cells.

Hormesis is the field of science which theorizes that when the body or cells are exposed to a mild physical, chemical or biological stress, such exposure results in an increased resistance to subsequent exposures to otherwise harmful doses of the same stressors. Those stressors include Reactive Oxygen Species (ROS). According to this theory, the same ROS that are produced during and/or as a result of exercise are thought to protect against ROS associated diseases. For example, the journal article entitled “Exercise and hormesis: oxidative stress-related adaptation for successful aging” by Radak et al. which is incorporated herein by reference in its entirety, discusses the hermetic effect of hydrogen peroxide to the extent that low concentrations of hydrogen peroxide increase Ca⁺⁺ in skeletal muscle whereas in contrast, large amounts of Ca⁺⁺ result in a sharp decrease in the amount of force outputted by the muscle. Similarly, Radak et al. saw an increase in proteasome activity without an associated increase in oxidative damage in the heart, subsequent to hydrogen peroxide administration to rats, which indicates that exercise may increase resistance against oxidative stress as well as boost the efficiency of repair processes (Radak et al. Exercise, oxidative stress and hormesis. Ageing Res. Rev. (2007) which is incorporated herein by reference in its entirety).

Accordingly, these ROS have been documented as signaling molecules which promote health and longevity (Ristow et al. How increased oxidative stress promotes longevity and metabolic health: The concept of mitochondrial hormesis (mitohormesis). Experimental Gerontology 45 (2010) 410-418 which is incorporated herein by reference in its entirety). It has also been suggested that ROS and redox signaling are involved in processes which help the body adapt to stressors (Moyer The Myth of Antioxidants. Scientific American February (2013) pp 62-67, which is incorporated herein by reference in its entirety).

On a cellular level, this is essentially the healthy tissue maintenance process: damaged cells are detected and repaired or replaced by healthy cells. This cellular repair and regeneration process is constantly taking place, millions of times an hour, in all parts of the body.

Furthermore, all of the molecular components found in these solutions are involved in a growing field of scientific investigation categorized as redox messaging and regulation of genes. Such molecular components, being a balanced set of reduced species (RS) and reactive oxygen species (ROS), are the same molecules and ions that mirror those found in biological systems and are intimately involved in the ability of the immune system to recognize, detect, eliminate and heal infected, damaged or mutated tissues in mammals.

Given the amount of stressors encountered by all forms of life, there is a need for better ways to help the body help itself in dealing with these stressors.

The invention comprises a composition of stabilized redox-signaling molecules that is particularly safe and suited for oral consumption. This composition is similar to that of a target composition of redox-signaling molecules that exists naturally inside a healthy human cell. The composition acts to enhance proper immune system function, to enhance the efficiency and production of the body's native antioxidants as well as to enhance the performance of intercellular communications involved in the maintenance of healthy tissues.

### SUMMARY

One method for producing a balanced foundational product to allow the body and immune system to function better, comprises first determining a balanced target mixture of redox-signaling molecules inherent to healthy cells and measuring the concentrations of the reactive molecules contained therein, usually with fluorescent indicators. These redox signaling molecules include ROS. ROS such as those occurring or being produced naturally in the mitochondria include O2- and hydrogen peroxide (Zhang et al. Mitochondrial reactive oxygen species-mediated signaling in endothelial cells. Heart May 2007 vol. 292 no. 5 H2023-H2031 which is incorporated herein in its entirety by reference.). These ROS can be measured as described in Dugan et al. Mitochondrial Production of Reactive Oxygen Species in Cortical Neurons Following Exposure to N-Methy-d-Aspartate. The Journal of Neuroscience, October 1995, 75(10): 6377-6366 and Lambert et al. Reactive Oxygen Species Production by Mitochondria. Mitochondrial DNA, Methods and Protocols, vol. 554 both of which are incorporated herein in their entireties by reference.

This target mixture is then replicated by the electrochemical method described herein in a process starting with a combination of pure water and salt (NaCl) that undergoes a specific electrochemical processing where the process parameters (temperature, flow, pH, power-source modulation and salt homogeneity and concentration) are varied to produce the ultimate specific target formulation.

The resulting formulation typically has less than about 10% of the recommended daily allowance (RDA) of sodium (usually between 115 mg to 131 mg of sodium per 4 fl. oz. serving) and a pH of between 7.0 and 8.5 with total chlorine less than 40 ppm. These ranges also make the product palatable (won't cause nausea) when taken in 8 oz or larger quantities. The sodium chloride concentration is a variable parameter that can be upwardly adjusted and still produce the desired target composition of the final composition mixture at the expense, of course, of becoming less palatable.

During the electrochemical process, to insure that the saline solution is well mixed, usually homogenizing means are included, such as a fluid circulation device to maintain flow aging stratification and homogeneity of the saline solution during electrolysis.

Next, the temperature and flow of the circulating saline is adjusted to a level to prevent production of chlorates and produce the desired relative concentrations of resulting chemical redox specie components during electrolysis using the apparatus and method disclosed in the parent application. The resultant redox specie components are then measured with the same indicators used to measure the balance of ROS and RS and the other chemical characteristics of the target mixture mentioned above. This process may involve an iterative process where the temperature, flow and other parameters are adjusted until a composition similar to that of the target mixture is achieved.

The resultant composition of reactive signaling molecules is stable with many of its components measurable using standard analytic methods. As discussed above, such signaling molecules are the same as those that are naturally produced inside of living cells and are measured using standard laboratory methods, such as the employment of certain fluorescent dyes that act as indicators. The concentration of some of the individual components of the composition is thus tested and verified in the laboratory.

For example, by regularly utilizing three standard fluorescent indicators, namely R-Phycoerythrin (R-PE), Aminophenyl fluorescein (APF) and Hydroxyphenyl fluorescein (HPF) their corresponding redox specie components can be tracked. Such fluorescent indicator molecules change brightness when they come into contact with specific redox specie. These indicator dyes are very resistant to false positives and are well studied. Such change in fluorescence is then measured using a fluorospectrometer. The change in fluorescence of these indicators quantifies the existence and relative concentration of their corresponding redox specie.

A combination of measurements from these indicators can be utilized to measure the concentration of reactive redox signaling molecules in the test composition and thereby the relative concentration of its major reactive molecular components. Several types of laboratory equipment and methods can also be employed to determine the composition of the proper target solution and that of the resultant electrolyzed composition. One such method is by the proper employment of a Nanodrop™ 3300 fluorospectrometer, made by Thermo Fischer Scientific, along with the R-PE, APF and HPF fluorescent dyes to measure the relative concentrations of reactive signaling molecules inside test compositions. Such measurements can then be compared to measurements taken from a desired target solution. Typically the test RSO Compound is measured along-side the desired target solution.

In one such method, the concentration and presence of such reactive molecules is verified when the three indicators, R-PE, APF and HPF show 1) that a 2 micro molar concentration of R-PE loses 5%-50% of its fluorescence 6 hours after a 1:1000 solution of the RS and ROS is added; 2) and R-PE measurements indicate the same fluorescence levels as a standard ROS generating solution of 0.2 to 1.0 mM AAPH, and 3) the APF measurements indicate the same relative amount as the target compound and 4) HPF measurements indicate a negligibly small reading and 5) the pH is between 7.2 and 7.5 and 6) the total chlorine is less that 35 ppm by weight.

Once the required electrolytic operating parameters are determined for producing the desired composition, the electrochemical device is then activated and adjusted to oxidize and reduce the saline solution in such a way as to produce a composition with similar concentration and mixture of reactive molecules as those present in the healthy target living cells.

The resultant composition is then administered orally or topically to a human as a supplement for the natural redox-signaling compounds formed inside the cells to enhance proper immune system function, to enhance the efficiency and production of the body's native antioxidants as well as to enhance the performance of intercellular communications involved in healthy tissue maintenance and athletic performance.

In summary, the composition of the redox-signaling composition is produced by utilizing an electrochemical process wherein the process parameters (temperature, flow, pH, power-source modulation and salt homogeneity and concentration) are varied until certain chemical indicators measure the same relative composition as compared to a target composition similar to that produced in the cells. The method and composition produced therefrom, thus provides a redox-signaling compound with reactive molecules that mimic those naturally occurring inside one's cells.

The resultant composition produced by the above method was tested to determine its efficacy by independent research. An in-vitro scientific investigation was done in conjunction with a prominent national laboratory to determine the bioactivity of this redox-signaling composition on eukaryotic cells in a controlled environment.

It has long been known that the electrolysis of fluids can result in useful products. Thus, various apparatus and methods have been proposed for electrolyzing saline solution, however, all of the previously available schemes present one or more drawbacks.

For example U.S. Pat. No. 7,691,249 teaches a method an apparatus for making electrolyzed water comprising an insulating end cap for a cylindrical electrolysis cell and is incorporated herein by reference in its entirety.

For example, U.S. Pat. Nos. 4,236,992 and 4,316,787 to Themy disclose an electrode, method and apparatus for electrolyzing dilute saline solutions to produce effective amounts of disinfecting agents such as chlorine, ozone and hydroxide ions. Both of these references are incorporated herein by reference in their entireties

U.S. Pat. No. 5,674,537, U.S. Pat. No. 6,117,285 and U.S. Pat. No. 6,007,686 also teach electrolyzed fluids and are now incorporated herein by reference in their entireties.

U.S. Pat. No. 4,810,344 teaches a water electrolyzing apparatus including a plurality of electrolysis devices, each comprising an electrolysis vessel having a cathode and an anode oppose to each other and an electrolysis diaphragm partitioning the space between both of the electrodes wherein the plurality of devices are connected in a series such that only one of the two ionized water discharge channels of the devices constitutes a water supply channel to the device a the succeeding stage and is incorporated herein by reference in its entirety.

U.S. Pat. No. 7,691,249 is now incorporated herein by reference in its entirety and is directed to a method and apparatus for making electrolyzed water.

U.S. Pat. No. 8,062,501 B2 is directed to a method for producing neutral electrolytic water containing OH, D2, HD and HDO as active elements and is incorporated herein by reference in its entirety.

Methods for treatment of physiological fluids using electrolyzed solutions are set forth in U.S. Pat. No. 5,334,383 which is now incorporated herein by reference in its entirety teaches an electrolyzed saline solution, properly made and administered in vivo, as effective in the treatment of various infections brought on by invading antigens and particularly viral infections.

U.S. Pat. No. 5,507,932 which is now incorporated herein by reference in its entirety teaches an apparatus for electrolyzing fluids.

In sum, the art teaches various methods of making electrolyzed fluids but none teaches the method described herein with its benefits of producing a stable solution with efficacy in benchmarking wellness.

In one embodiment, the invention is directed to a method of benchmarking wellness of a subject comprising measuring the rate of muscle glycogen depletion in the subject after exercise and comparing said rate of muscle glycogen depletion to the rate of muscle glycogen depletion of a known standard.

In another embodiment, the invention is directed to a method of benchmarking wellness wherein the known standard is the post-exercise average rate of muscle glycogen depletion for a known or given population.

In another embodiment, the invention is directed to a method of benchmarking wellness wherein the difference in the rate of muscle glycogen depletion between the subject and the known standard is measured as a percentage of the known standard.

In still another embodiment, the invention is directed to a method of benchmarking wellness wherein there is a decrease in the rate of muscle glycogen depletion in the subject compared to the known standard, and wherein the decrease in the rate of muscle glycogen depletion is from 1-35%.

In one instance, the invention is directed to a method of benchmarking wellness further comprising administering to the subject a composition comprising at least one RXN wherein the RXN is selected from the group consisting of reduced species (RS) and reactive oxygen species (ROS).

In another instance, the invention is directed to a method of benchmarking wellness wherein the composition comprising at least one RXN is made by electrolyzing a homogenous and well mixed solution of saline and water.

In still another instance, the invention is directed to a method of benchmarking wellness wherein the temperature, flow and electrical current are adjusted during the process of electrolyzing.

In one embodiment, the invention is directed to a method of benchmarking wellness wherein the temperature at the time of electrolyzing is between 30-100° F.

In another embodiment, the invention is directed to a method of benchmarking wellness wherein the voltage drops to zero at least once per second during the process of electrolyzing and further wherein the voltage remains positive during the process of electrolyzing.

In another embodiment, the invention is directed to a method of benchmarking wellness wherein the composition comprises at least one ROS and the at least one ROS includes a superoxide and the superoxide is *O2-.

In still another embodiment, the invention is directed to a method of benchmarking wellness wherein the presence of the superoxide is detectable for at least 10 years.

In one instance, the invention is directed to a method of benchmarking wellness wherein the at least one reduced species (RS) includes HOCl, NaClO, O2, H2, H+, ClO, Cl2, H2O2 or mixtures thereof and the at least one reactive oxygen species (ROS) includes O2-, HO2, Cl—, H—, *OCl, O3, *O2-, OH— or mixtures thereof.

In another instance, the invention is directed to a method of benchmarking wellness wherein the composition comprises hypochlorous acid or a salt thereof.

In another instance, the invention is directed to a method of benchmarking wellness wherein at least 60% of the at least one reactive oxygen species (ROS) is present in the composition after 1 year.

In still another instance, the invention is directed to a method of benchmarking wellness wherein at least 98% of the at least one reactive oxygen species (ROS) is present in the composition after 1 year.

In still another instance, the invention is directed to a method of benchmarking wellness wherein at least 65% of the at least one reactive oxygen species (ROS) is present in the composition after 10 years.

In yet another embodiment, the invention is directed to a method of benchmarking wellness wherein at least 100% of the at least one reactive oxygen species (ROS) is present in the composition after 10 years.

In yet another embodiment, the invention is directed to a method of benchmarking wellness wherein the at least one reactive oxygen species (ROS) has a half-life of about 24 years.

In one instance, the invention is directed to a method of benchmarking wellness wherein the at least one reactive oxygen species (ROS) has a half-life of greater than about 24 years.

In yet another instance, the invention is directed to a method of benchmarking wellness wherein the at least one reactive oxygen species (ROS) is hypochlorous acid or a salt thereof.

In yet another embodiment, the invention is directed to a method of predicting physical or athletic endurance comprising testing the p-ACC of an individual and comparing the level of p-ACC expression to the average p-ACC of a known population.

### BRIEF DESCRIPTION OF THE DRAWINGS

FIG. 1 is a flow chart of a process as described herein.

FIG. 2 illustrates an example diagram of the generation of various molecules at the electrodes. The molecules written between the electrodes depict the initial reactants and those on the outside of the electrodes depict the molecules/ions produced at the electrodes and their electrode potentials.

FIG. 3 illustrates a plan view of a process and system for producing a composition according to the present description.

FIG. 4 illustrates an example system for preparing water for further processing into a composition described herein.

FIG. 5 illustrates a Cl35 spectrum of NaCl, NaClO solution at a pH of 12.48, and a composition described herein (the composition is labeled “ASEA”).

FIG. 6 illustrates a 1H NMR spectrum of a composition of the present disclosure.

FIG. 7 illustrates a 31P NMR spectrum of DIPPMPO combined with a composition described herein.

FIG. 8 illustrates a mass spectrum showing a parent peak and fragmentation pattern for DIPPMPO with m/z peaks at 264, 222, and 180.

FIG. 9 illustrates oxygen/nitrogen ratios for a composition described herein compared to water and NaClO (the composition is labeled “ASEA”).

FIG. 10 illustrates chlorine/nitrogen ratios for a composition described herein compared to water and NaClO (the composition is labeled “ASEA”).

FIG. 11 illustrates ozone/nitrogen ratios for a composition described herein compared to water and NaClO (the composition is labeled “ASEA”).

FIG. 12 illustrates the carbon dioxide to nitrogen ratio of a composition as described herein compared to water and NaClO (the composition is labeled “ASEA”).

FIG. 13 illustrates an EPR splitting pattern for a free electron.

FIG. 14 is a perspective view of a first presently preferred embodiment of the present invention.

FIG. 15 is a detailed top view of the electrode assembly represented in FIG. 14.

FIG. 15A is a side cross sectional view of the electrode assembly taken along line 3-3 in FIG. 15.

FIG. 16 is a block diagram of a second presently preferred embodiment of the present invention.

FIG. 17 is a top view of an electrode assembly preferred for use in the apparatus represented in FIG. 16.

FIG. 18 is a cross sectional view taken along line 6-6 of FIG. 17.

FIG. 19 Illustrates a block diagram of a power source.

FIG. 20 Illustrates a block diagram of another power source.

FIG. 21 is a chart of the relative fluorescence of various compositions.

FIG. 22 is a graph of the decay rate of superoxide over a period of 1 year.

FIG. 23 is a graph showing the comparison of the decay rates of superoxide when the mixture is stored in a bottle and when the mixture is stored in a pouch.

FIG. 24 is a graph of the Expt. 5f07 ROS Assay.

FIG. 25 is a graph of an Intraassay Variation Using Two Levels of AAPH.

FIG. 26 is a study design to measure the effects of a product comprising ROS and RXNs on endurance performance in mice.

FIG. 27 is a comparison of the effects of a product comprising ROS and RXNs on endurance run time in mice.

FIG. 28 is a comparison of the effects of a product comprising ROS and RXNs on the rate of muscle glycogen depletion in mice.

FIG. 29 is a comparison of the effects of a product comprising ROS and RXNs on phosphorylated acetyl-CoA carboxylate (p-ACC or phosphor/pan-ACC) in mice.

FIG. 30 shows a typical data set displaying graphical analysis methods used to determine Ventilatory Threshold (VT).

### DETAILED DESCRIPTION OF THE INVENTION

Described herein are compositions including fluids that generally include at least one redox signaling agent (RXN) and methods of using such compositions. RXNs can include, but are not limited to superoxides: O₂*—, HO₂*; hypochlorites: OCl—, HOCl, NaOCl; hypochlorates: HClO₂, ClO₂, HClO₃, HClO₄; oxygen derivatives: O₂, O₃, O₄*—, 1O; hydrogen derivatives: H₂, H—; hydrogen peroxide: H₂O₂; hydroxyl free Radical: OH*—; ionic compounds: Na+, Cl—, H+, OH—, NaCl, HCl, NaOH; chlorine: Cl₂; water clusters: n*H₂O— induced dipolar layers around ions and combinations thereof. Some RXNs are electron acceptors (RS) and include HOCl, NaClO, O₂, H₂, H+, ClO, Cl₂, H₂O₂ and some are electron donors and include O₂—, HO₂, Cl—, H—, *OCl, O₃, *O₂— and OH—.

RXNs include hypochlorous acid and salts thereof. The amount of hypochlorous acid can be predetermined by adjusting the amount of salt used, the temperature maintained during the electrochemical or electrolysis process, the time spent for processing and other parameters. Hypochlorous acid and/or salts thereof can be present in an amount of from 1 to 1000 ppm. Preferably, hypochlorous acid and/or salts thereof can be present in an amount of from 1 to 500 ppm. More preferably, hypochlorous acid and/or salts thereof can be present in an amount of from 1 to 100 ppm. Hypochlorous acid and/or salts thereof can be present for example in an amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 ppm.

Methods of producing the disclosed compositions can include one or more of the steps of (1) preparation of an ultra-pure homogeneous solution of sodium chloride in water, (2) temperature control and flow regulation through a set of inert catalytic electrodes and (3) a modulated electrolytic process that results in the formation of such stable molecular moieties and complexes; the RS and ROS. In one embodiment, such a process includes all these steps.

A general example of one such method of making therapeutic compositions is described as comprising: electrolyzing salinated water having a salt concentration of about 2.8 g NaCl/L, using a set of electrodes with an amperage of about 3 amps, to form an antifungal composition, wherein the water is at or below room temperature during 3 minutes of electrolyzing.

Another general example of one such method of making therapeutic compositions is described as comprising: electrolyzing salinated water having a salt concentration of about 9.1 g NaCl/L, using a set of electrodes with an amperage of about 3 amps, to form an antifungal composition, wherein the water is at or below room temperature during 3 minutes of electrolyzing.

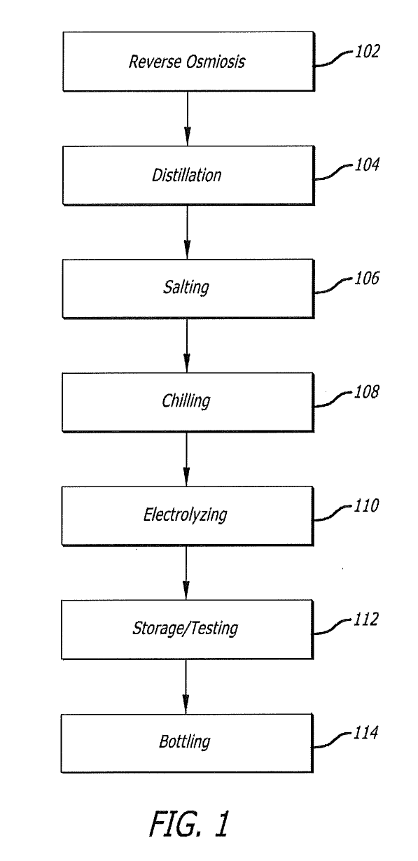

Water can be supplied from a variety of sources, including but not limited to municipal water, filtered water, nanopure water, or the like. With this in mind, a step in such a process is shown in FIG. 1. 100 is an optional reverse osmosis procedure 102.

The reverse osmosis process can vary, but can provide water having a total dissolved solids content of less than about 10 ppm, about 9 ppm, about 8 ppm, about 7 ppm, about 6 ppm, about 5 ppm, about 4 ppm, about 3 ppm, about 2 ppm, about 1 ppm, or the like.

The reverse osmosis process can be performed at a temperature of about 5° C., about 10° C., about 15° C., about 20° C., about 25° C., about 30° C., about 35° C., or the like. The reverse osmosis step can be repeated as needed to achieve a particular total dissolved solids level. Whether the optional reverse osmosis step is utilized, an optional distillation step 104 can be performed.

Other means of reducing contaminants include filtration and/or purification such as by utilizing deionization, carbon filtration, double-distillation, electrodeionization, resin filtration such as with Milli-Q purification, microfiltration, ultrafiltration, ultraviolet oxidation, electrodialysis, or combinations thereof.

The distillation process can vary, but can provide water having a total dissolved solids content of less than about 5 ppm, about 4 ppm, about 3 ppm, about 2 ppm, about 1 ppm, about 0.9 ppm, about 0.8 ppm, about 0.7 ppm, about 0.6 ppm, about 0.5 ppm, about 0.4 ppm, about 0.3 ppm, about 0.2 ppm, about 0.1 ppm, or the like. The temperature of the distillation process can be performed at a temperature of about 5° C., about 10° C., about 15° C., about 20° C., about 25° C., about 30° C., about 35° C., or the like.

The distillation step can be repeated as needed to achieve a particular total dissolved solids level. After water has been subjected to reverse osmosis, distillation, both, or neither, the level of total dissolved solids in the water can be less than about 5 ppm, about 4 ppm, about 3 ppm, about 2 ppm, about 1 ppm, about 0.9 ppm, about 0.8 ppm, about 0.7 ppm, about 0.6 ppm, about 0.5 ppm, about 0.4 ppm, about 0.3 ppm, about 0.2 ppm, about 0.1 ppm, or the like.

The reverse osmosis, distillation, both, or neither, can be preceded by a carbon filtration step.

Purified water can be used directly with the systems and methods described herein.

In one embodiment, contaminants can be removed from a commercial source of water by the following procedure: water flows through an activated carbon filter to remove the aromatic and volatile contaminants and then undergoes Reverse Osmosis (RO) filtration to remove dissolved solids and most organic and inorganic contaminants. The resulting filtered RO water can contain less than about 8 ppm of dissolved solids. Most of the remaining contaminants can be removed through a distillation process, resulting in dissolved solid measurements less than 1 ppm. In addition to removing contaminants, distillation may also serve to condition the water with the correct structure and Oxidation Reduction Potential (ORP) to facilitate the oxidative and reductive reaction potentials on the platinum electrodes in the subsequent electro-catalytic process.

The saline generally should be free from contaminants, both organic and inorganic, and homogeneous down to the molecular level. In particular, metal ions can interfere with the electro-catalytic surface reactions, and thus it may be helpful for metals to be avoided. In one embodiment, a brine solution is used to salinate the water. The brine solution can have a NaCl concentration of about 540 g NaCl/gal, such as 537.5 g NaCl/gal.

After water has been subjected to reverse osmosis, distillation, both or neither, or any other purification step as described herein, a salt is added to the water in a salting step 106 of FIG. 1. The salt can be unrefined, refined, caked, de-caked, or the like. In one embodiment, the salt is sodium chloride (NaCl). In some embodiments, the salt can include an additive. Salt additives can include, but are not limited to potassium iodide, sodium iodidie, sodium iodate, dextrose, sodium fluoride, sodium ferrocyanide, tricalcium phosphate, calcium carbonate, magnesium carbonate, fatty acids, magnesium oxide, silicone dioxide, calcium silicate, sodium aluminosilicate, calcium aluminosilicate, ferrous fumarate, iron, or folic acid. Any of these additives can be added at this point or at any point during the described process. For example, the above additives can be added just prior to bottling.

In another embodiment, the process can be applied to any ionic, soluble salt mixture, especially with those containing chlorides. In addition to NaCl, other non-limiting examples include LiCl, HCl, CuCl2, CuSO4, KCl, MgCl, CaCl2, sulfates and phosphates. For example, strong acids such as sulfuric acid (H2SO4), and strong bases such as potassium hydroxide (KOH), and sodium hydroxide (NaOH) are frequently used as electrolytes due to their strong conducting abilities. Preferably the salt is sodium chloride (NaCl). A brine solution can be used to introduce the salt into the water. The amount of brine or salt needs will be apparent to one of ordinary skill in the art.

Salt can be added to water in the form of a brine solution. To mix the brine solution, a physical mixing apparatus can be used or a circulation or recirculation can be used. In one embodiment, pure pharmaceutical grade sodium chloride is dissolved in the prepared distilled water to form a 15 wt % sub-saturated brine solution and continuously re-circulated and filtered until the salt has completely dissolved and all particles >0.1 microns are removed. This step can take several days. The filtered, dissolved brine solution is then injected into tanks of distilled water in about a 1:352 ratio (salt:water) in order to form a 0.3% saline solution. In one embodiment, a ratio 10.75 g of salt per 1 gallon of water can be used to form the composition. In another embodiment, 10.75 g of salt in about 3-4 g of water, such as 3,787.5 g of water can be used to form the composition. This solution then can be allowed to re-circulate and diffuse until homogeneity at the molecular scale has been achieved. The brine solution can have a NaCl concentration of about 540 g NaCl/gal, such as 537.5 g NaCl/gal.

Brine can then be added to the previously treated water or to fresh untreated water to achieve a NaCl concentration of between about 1 g NaCl/gal water and about 25 g NaCl/gal water, between about 8 g NaCl/gal water and about 12 g NaCl/gal water, or between about 4 g NaCl/gal water and about 16 g NaCl/gal water. In a preferred example, the achieved NaCl concentration is 2.8 g/L of water. In another preferred example, the achieved NaCl concentration is 9.1 g/L of water. Once brine is added to water at an appropriate amount, the solution can be thoroughly mixed. The temperature of the liquid during mixing can be at room temperature or controlled to a desired temperature or temperature range.

To mix the solution, a physical mixing apparatus can be used or a circulation or recirculation can be used. The salt solution can then be chilled in a chilling step 108 of FIG. 1.

For large amounts of composition, various chilling and cooling methods can be employed. For example cryogenic cooling using liquid nitrogen cooling lines can be used. Likewise, the solution can be run through propylene glycol heat exchangers to achieve the desired temperature. The chilling time can vary depending on the amount of liquid, the starting temperature and the desired chilled temperature.

Products from the anodic reactions can be effectively transported to the cathode to provide the reactants necessary to form the stable complexes on the cathode surfaces. Maintaining a high degree of homogeneity in the fluids circulated between the catalytic surfaces can also be helpful. A constant flow of about 2-8 mL/cm2 per sec can be used, with typical mesh electrode distances 2 cm apart in large tanks. This flow can be maintained, in part, by the convective flow of gasses released from the electrodes during electrolysis.

The mixed solution, chilled or not, can then undergo electrochemical processing through the use of at least one electrode in an electrolyzing step 110 of FIG. 1. Each electrode can be or include a conductive metal. Metals can include, but are not limited to copper, aluminum, titanium, rhodium, platinum, silver, gold, iron, a combination thereof or an alloy such as steel or brass. The electrode can be coated or plated with a different metal such as, but not limited to aluminum, gold, platinum or silver. In an embodiment, each electrode is formed of titanium and plated with platinum. The platinum surfaces on the electrodes by themselves can be optimal to catalyze the required reactions. Rough, double layered platinum plating can assure that local “reaction centers” (sharply pointed extrusions) are active and that the reactants not make contact with the underlying electrode titanium substrate.

In one embodiment, rough platinum-plated mesh electrodes in a vertical, coaxial, cylindrical geometry can be optimal, with, for example, not more than 2.5 cm, not more than 5 cm, not more than 10 cm, not more than 20 cm, or not more than 50 cm separation between the anode and cathode. The amperage run through each electrode can be between about 2 amps and about 15 amps, between about 4 amps and about 14 amps, at least about 2 amps, at least about 4 amps, at least about 6 amps, or any range created using any of these values. In one embodiment, 7 amps is used with each electrode. In one example, 1 amp is run through the electrodes. In one example, 2 amps are run through the electrodes. In one example, 3 amps are run through the electrodes. In one example, 4 amps are run through the electrodes. In one example, 5 amps are run through the electrodes. In one example, 6 amps are run through the electrodes. In one example, 7 amps are run through the electrodes. In a preferred example, 3 amps are run through the electrodes.

The amperage can be running through the electrodes for a sufficient time to electrolyze the saline solution. The solution can be chilled during the electrochemical process. The solution can also be mixed during the electrochemical process. This mixing can be performed to ensure substantially complete electrolysis.

Electric fields between the electrodes can cause movement of ions. Negative ions can move toward the anode and positive ions toward the cathode. This can enable exchange of reactants and products between the electrodes. In some embodiments, no barriers are needed between the electrodes.

After amperage has been run through the solution for a sufficient time, an electrolyzed solution is created. The solution can be stored and or tested for particular properties in storage/testing step 112 of FIG. 1. In one embodiment, the homogenous saline solution is chilled to about 4.8±0.5° C. Temperature regulation during the entire electro-catalytic process is typically required as thermal energy generated from the electrolysis process itself may cause heating. In one embodiment, process temperatures at the electrodes can be constantly cooled and maintained at about 4.8° C. throughout electrolysis.

After amperage has been run through the solution for a sufficient time, an electrolyzed solution is created with beneficial properties, such as antifungal properties. The solution can have a pH of about 7.4. In some embodiments, the pH is greater than 7.3. In some embodiments, the pH is not acidic. In other embodiments, the solution can have a pH less than about 7.5. The pH may not be basic. The solution can be stored and or tested for particular properties in a storage/testing step 112 of FIG. 1.

The end products of this electrolytic process can react within the saline solution to produce many different chemical entities. The compositions and composition described herein can include one or more of these chemical entities, known as redox signaling agents or RXNs.

The chlorine concentration of the electrolyzed solution can be between about 5 ppm and about 34 ppm, between about 10 ppm and about 34 ppm, or between about 15 ppm and about 34 ppm. In one embodiment, the chlorine concentration is about 32 ppm.

The saline concentration in the electrolyzed solution can be, for example, between about 0.10% w/v and about 0.20% w/v, between about 0.11% w/v and about 0.19% w/v, between about 0.12% w/v and about 0.18% w/v, between about 0.13% w/v and about 0.17% w/v, or between about 0.14% w/v and about 0.16% w/v.

The composition can then be bottled in a bottling step 114 of FIG. 1. The composition can be bottled in plastic bottles having volumes of about 4 oz, about 8 oz, about 16 oz, about 32 oz, about 48 oz, about 64 oz, about 80 oz, about 96 oz, about 112 oz, about 128 oz, about 144 oz, about 160 oz, or any range created using any of these values. The plastic bottles can also be plastic squeezable pouches having similar volumes. In one embodiment, plastic squeezable pouches can have one way valves to prevent leakage of the composition, for example, during athletic activity.

During bottling, solution from an approved batch can be pumped through a 10 micron filter (e.g., polypropylene) to remove any larger particles from tanks, dust, hair, etc. that might have found their way into the batch. In other embodiments, this filter need not be used. Then, the solution can be pumped into the bottles, the overflow going back into the batch.

Bottles generally may not contain any dyes, metal specks or chemicals that can be dissolved by acids or oxidating agents. The bottles, caps, bottling filters, valves, lines and heads used can be specifically be rated for acids and oxidating agents. Caps and with organic glues, seals or other components sensitive to oxidation may be avoided, as these could neutralize and weaken the product over time.

The bottles and pouches used herein can aid in preventing decay of free radical species found within the compositions. In other embodiments, the bottles and pouches described do not further the decay process. In other words, the bottles and pouches used can be inert with respect to the radical species in the composition s. In one embodiment, a container (e.g., bottle and/or pouch) can allow less than about 10% decay/month, less than about 9% decay/month, less than about 8% decay/month, less than about 7% decay/month, less than about 6% decay/month, less than about 5% decay/month, less than about 4% decay/month, less than about 3% decay/month, less than about 2% decay/month, less than about 1% decay/month, between about 10% decay/month and about 1% decay/month, between about 5% decay/month and about 1% decay/month, about 10% decay/month, about 9% decay/month, about 8% decay/month, about 7% decay/month, about 6% decay/month, about 5% decay/month, about 4% decay/month, about 3% decay/month, about 2% decay/month, or about 1% decay/month of free radicals in the composition. In one embodiment, a bottle can only result in about 3% decay/month of superoxide. In another embodiment, a pouch can only result in about 4% decay/month of superoxide.

A direct current, DC, power source is used to electrolyze water.

The variables of voltage, amps, frequency, time and current required depend on the compound and/or ion themselves and their respective bond strengths. To that end, the variables of voltage, amps, frequency, time and current are compound and/or ion dependent and are not limiting factors. That notwithstanding, the voltage used can be less than 40V, such as 30V or 20V or 10V or any voltage in between. The voltage can also modulate and at any time vary within a range of from 1 to 40V or from 10 to 30V or from 20 to 30V. In one embodiment, the voltage can range during a single cycle of electrolyzing. The range can be from 1 to 40V or from 10 to 30V or from 20 to 30V. These ranges are non-limiting but are shown as examples.

Waveforms with an AC ripple also referred to as pulse or spiking waveforms include: any positive pulsing currents such as pulsed waves, pulse train, square wave, sawtooth wave, spiked waveforms, pulse-width modulation (PWM), pulse duration modulation (PDM), single phase half wave rectified AC, single phase full wave rectified AC or three phase full wave rectified for example.

A bridge rectifier may be used. Other types of rectifiers can be used such as Single-phase rectifiers, Full-wave rectifiers, Three-phase rectifiers, Twelve-pulse bridge, Voltage-multiplying rectifiers, filter rectifier, a silicon rectifier, an SCR type rectifier, a high-frequency (RF) rectifier, an inverter digital-controller rectifier, vacuum tube diodes, mercury-arc valves, solid-state diodes, silicon-controlled rectifiers and the like. Pulsed waveforms can be made with a transistor regulated power supply, a dropper type power supply, a switching power supply and the like.

A transformer may be used. Examples of transformers that can be used include center tapped transformers, Autotransformer, Capacitor voltage transformer, Distribution transformer, power transformer, Phase angle regulating transformer, Scott-T transformer, Polyphase transformer, Grounding transformer, Leakage transformer, Resonant transformer, Audio transformer, Output transformer, Laminated core Toroidal Autotransformer, Variable autotransformer, Induction regulator, Stray field transformer, Polyphase transformer, Grounding transformer, Leakage transformers, Resonant transformer, Constant voltage transformer, Ferrite core Planar transformer Oil cooled transformer, Cast resin transformer, Isolating transformer, Instrument transformer, Current transformer, Potential transformer Pulse transformer transformer Air-core transformer, Ferrite-core transformer, Transmission-line transformer, Balun Audio transformer, Loudspeaker transformer, Output transformer, Small signal transformer, Interstage coupling transformers, Hedgehog or Variocoupler.

Pulsing potentials in the power supply of the production units can also be built in. Lack of filter capacitors in the rectified power supply can cause the voltages to drop to zero many times per second, resulting in a hard spike when the alternating current in the house power lines changes polarity. This hard spike, under Fourier transform, can emit a large bandwidth of frequencies. In essence, the voltage is varying from high potential to zero 120 times a second. In other embodiments, the voltage can vary from high potential to zero about 1,000 times a second, about 500 times a second, about 200 times a second, about 150 times a second, about 120 times a second, about 100 times a second, about 80 times a second, about 50 times a second, about 40 times a second, about 20 times a second, between about 200 times a second and about 20 times a second, between about 150 times a second and about 100 times a second, at least about 100 times a second, at least about 50 times a second, or at least about 120 times a second. This power modulation can allow the electrodes sample all voltages and also provides enough frequency bandwidth to excite resonances in the forming molecules themselves. The time at very low or zero voltages can also provide an environment of low electric fields where ions of similar charge can come within close proximity to the electrodes. All of these factors together can provide a possibility for the formation of stable complexes capable of generating and preserving ROS free radicals. To that end, the voltage can drop to zero at least 1 time per second or greater than 1 time per second, for example 2 times or 3 times or 4 times or any whole number of times up to a whole number in the thousands. Combinations are also possible. For example the frequency can change or fluctuate during each second such that the period between each drop in voltage or each spike in voltage is constantly changing. As mentioned elsewhere, and without being bound by theory, it is believed that the electricity alters the state of some of the ions/compounds. This alteration results in the pushing of electrons out of their original orbit and/or spin state into a higher energy state and/or a single spin state. This electrolysis provides the energy to form free radicals which are ultimately formed during a multi-generational cycling of reactants and products during the electrolysis process. In other words, compounds and/or ions are initially electrolyzed so that the products that are formed are then themselves reacted with other compounds and/or ions and/or gas to form a second generation of reactants and products. This generational process then happens again so that the products from the second generation react with other compounds and/or ions in solution when the voltage spikes again.

The ions have time to migrate in the solution when the voltage drops to zero. Therefore, each time the voltage drops to zero, new interactions between the ionic components in solution are possible. The more opportunities the ions have to interact, the faster the generational process can be effectuated. Along these lines, the shorter the period, the more opportunities the ionic components will have to interact.

A form of DC current is required, however, the DC current may include waveforms that have AC characteristics. Waveforms with an alternating current (AC) ripple can be used to provide power to the electrodes. Such an AC ripple can also be referred to as pulse or spiking waveforms and include: any positive pulsing currents such as pulsed waves, pulse train, square wave, sawtooth wave, pulse-width modulation (PWM), pulse duration modulation (PDM), single phase half wave rectified AC, single phase full wave rectified AC or three phase full wave rectified for example.

A bridge rectifier may be used. Other types of rectifiers can be used such as Single-phase rectifiers, Full-wave rectifiers, Three-phase rectifiers, Twelve-pulse bridge, Voltage-multiplying rectifiers, filter rectifier, a silicon rectifier, an SCR type rectifier, a high-frequency (RF) rectifier, an inverter digital-controller rectifier, vacuum tube diodes, mercury-arc valves, solid-state diodes, silicon-controlled rectifiers and the like. Pulsed waveforms can be made with a transistor regulated power supply, a dropper type power supply, a switching power supply and the like.

This pulsing waveform model can be used to stabilize superoxides, hydroxyl radicals and OOH* from many different components and is not limited to any particular variable such as voltage, amps, frequency, flux (current density) or current. The variables are specific to the components used. For example, water and NaCl can be combined which provide molecules and ions in solution. A 60 Hz current can be used, meaning that there are 60 cycles/120 spikes in the voltage (V) per second or 120 times wherein the V is 0 each second. When the V goes down to 0 it is believe that the 0 V allows for ions to drift apart/migrate and reorganize before the next increase in V. It is theorized that this spiking in V allows for and promotes a variable range of frequencies influencing many different types of compounds and/or ions so that this process occurs.

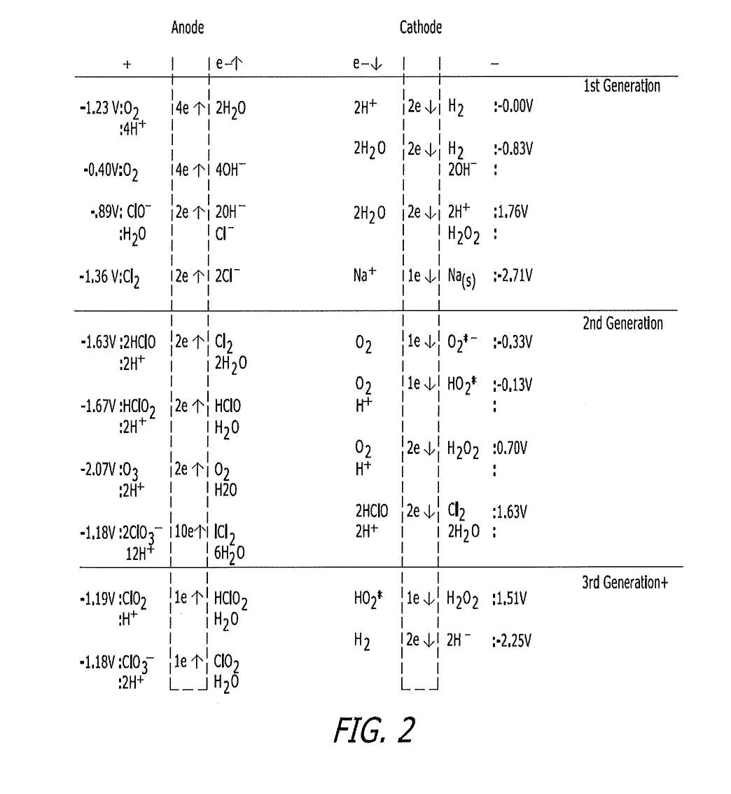

In one embodiment, periodic moments of 0 volts are required. Again, when the V goes down to 0 it is believe that the 0 V allows for ions to drift apart/migrate and reorganize before the next increase in V. Therefore, without being bound to theory, it is believed that this migration of ions facilitates the 1^{st}, 2^{nd}, and 3^{rd} generations of species as shown in FIG. 2. Stabilized superoxides, such as O₂^{*-}, are produced by this method.

In another embodiment, the V is always either 0 V or a positive potential.

Diodes may also be used. The V may drop to 0 as many times per second as the frequency is adjusted. As the frequency is increased the number of times the V drops is increased.

When the ions are affected by the electricity from the electrodes, they change. Without being bound by theory, it is believed that the electricity alters the state of some of the ions/compounds. This alteration results in the pushing of electrons out of their original orbit and/or spin state into a higher energy state and/or a single spin state. This electrolysis provides the energy to form free radicals which are ultimately formed during a multi-generational cycling of reactants and products during the electrolysis process. In other words, compounds and/or ions are initially electrolyzed so that the products that are formed are then themselves reacted with other compounds and/or ions and/or gas to form a second generation of reactants and products. This generational process then happens again so that the products from the second generation react with other compounds and/or ions in solution when the voltage spikes again.

The redox potential can be about 840 mV.

The frequency can be from 1 Hz to infinity or to 100 MHz. Preferably, the frequency is from 20 Hz to 100 Hz. More preferably, the frequency is from 40 Hz to 80 Hz. Most preferably, the frequency is 60 Hz. In one embodiment, the frequency is any of 21 Hz, 22 Hz, 23 Hz, 24 Hz, 25 Hz, 26 Hz, 27 Hz, 28 Hz, 29 Hz, 30 Hz, 31 Hz, 32 Hz, 33 Hz, 34 Hz, 35 Hz, 36 Hz, 37 Hz, 38 Hz, 39 Hz, 40 Hz, 41 Hz, 42 Hz, 43 Hz, 44 Hz, 45 Hz, 46 Hz, 47 Hz, 48 Hz, 49 Hz, 50 Hz, 51 Hz, 52 Hz, 53 Hz, 54 Hz, 55 Hz, 56 Hz, 57 Hz, 58 Hz, 59 Hz, 60 Hz, 61 Hz, 62 Hz, 63 Hz, 64 Hz, 65 Hz, 66 Hz, 67 Hz, 68 Hz, 69 Hz, 70 Hz, 71 Hz, 72 Hz, 73 Hz, 74 Hz, 75 Hz, 76 Hz, 77 Hz, 78 Hz, 79 Hz or 80 Hz. In one embodiment, the frequency is variable such that it varies from 1 Hz to 100 Hz or from 20 Hz to 80 Hz or from 30 Hz to 70 Hz or from 40 Hz to 60 Hz or combinations thereof.

Again referencing FIG. 2, FIG. 2 illustrates an example diagram of the generation of various molecules at the electrodes, the molecules written between the electrodes depict the initial reactants and those on the outside of the electrodes depict the molecules/ions produced at the electrodes and their electrode potentials. The diagram is broken into generations where each generation relies on the products of the subsequent generations.

The temperature maintained during the electrochemical process can vary and be adjusted as desired. Preferably, the temperature maintained during the electrochemical process can be from 30 to 100° F. More preferably, the temperature maintained during the electrochemical or electrolysis process can be 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100° F.

The end products of this electrolytic process can react within the saline solution to produce many different chemical entities. The compositions described herein can include one or more of these chemical entities. These end products can include, but are not limited to superoxides: O2*-, HO2*; hypochlorites: OCl—, HOCl, NaOCl; hypochlorates: HClO2, ClO2, HClO3, HClO4; oxygen derivatives: O2, O3, O4*-, 1O; hydrogen derivatives: H2, H—; hydrogen peroxide: H2O2; hydroxyl free Radical: OH*—; ionic compounds: Na+, Cl—, H+, OH—, NaCl, HCl, NaOH; chlorine: Cl2; and water clusters: n*H2O— induced dipolar layers around ions, several variations.

In one embodiment, the composition can include at least one species such as O2, H2, Cl2, OCl—, HOCl, NaOCl, HClO2, ClO2, HClO3, HClO4, H2O2, Na+, Cl—, H+, H, OH—, O3, O4*, 1O, OH*—, HOCl—O2*-, HOCl—O3, O2*, HO2*, NaCl, HCl, NaOH, water clusters, or a combination thereof.

In one embodiment, the composition can include at least one species such as H2, Cl2, OCl—, HOCl, NaOCl, HClO2, ClO2, HClO3, HClO4, H2O2, O3, O4*, 1O2, OH*—, HOCl—O2*-, HOCl—O3, O2*, HO2*, water clusters, or a combination thereof.

In one embodiment, the composition can include at least one species such as HClO3, HClO4, H2O2, O3, O4*, 1O2, OH*—, HOCl—O2*-, HOCl—O3, O2*, HO2*, water clusters, or a combination thereof.

In one embodiment, the composition can include at least O2* and HOCl.

In one embodiment, the composition can include O2. In one embodiment, the composition can include H2. In one embodiment, the composition can include Cl2. In one embodiment, the composition can include OCl—. In one embodiment, the composition can include HOCl. In one embodiment, the composition can include NaOCl. In one embodiment, the composition can include HClO2. In one embodiment, the composition can include ClO2. In one embodiment, the composition can include HClO3. In one embodiment, the composition can include HClO4. In one embodiment, the composition can include H2O2. In one embodiment, the composition can include Na+. In one embodiment, the composition can include Cl2. In one embodiment, the composition can include H+. In one embodiment, the composition can include H. In one embodiment, the composition can include OH—. In one embodiment, the composition can include O3. In one embodiment, the composition can include O4*. In one embodiment, the composition can include 1O2. In one embodiment, the composition can include OH*—. In one embodiment, the composition can include HOCl—O2*-. In one embodiment, the composition can include HOCl—O3. In one embodiment, the composition can include O2*. In one embodiment, the composition can include HO2*. In one embodiment, the composition can include NaCl. In one embodiment, the composition can include HCl. In one embodiment, the composition can include NaOH. In one embodiment, the composition can include water clusters. Embodiments can include combinations thereof.

In some embodiments, hydroxyl radicals can be stabilized in the composition by the formation of radical complexes. The radical complexes can be held together by hydrogen bonding. Another radical that can be present in the composition is an OOH* radical. Still other radical complexes can include a nitroxyl-peroxide radical (HNO—HOO*) and/or a hypochlorite-peroxide radical (HOCl—HOO*).

Certain RXNs as used herein are considered stable. As used herein, the term “stable” may refer to chemical stability and/or physical stability. When referring to chemical stability, stable means the ability of a compound to maintain its chemical identity over time and/or be measured over time. Accordingly, stability implies the ability of a chemical species to resist oxidation, hydrolysis or other degradation, for example. When referring to physical stability, stable means the ability of a composition to maintain consistent physical properties over time. The ability of a composition to maintain a consistent disintegration time over time is exemplary of physical stability.

Reactive Oxygen Species

Stable oxygen radicals can remain stable for about 3 months, about 6 months, about 9 months, about 12 months, about 15 months, about 18 months, about 21 months, between about 9 months and about 15 months, between about 12 months and about 18 months, at least about 9 months, at least about 12 months, at least about 15 months, at least about 18 months, about 24 months, about 30 months, about 50 months, about 100 months, about 200 months, about 300 months, about 400 months, about 500 months, about 1000 months, about 2000 months, or longer.

Reactive oxygen species can remain present in the solution for at least 3 months, at least 6 months, at least 9 months, at least 12 months, at least 15 months, at least 18 months, at least 21 months, at least 30 months, at least 50 months, at least 100 months, at least 200 months, at least 300 months, at least 400 months, at least 500 months, at least 1000 months, at least 2000 months, or longer. Reactive oxygen species can remain present in the solution for between 3 months and 2000 months, between 100 months and 1000 months, between 500 months and 800 months.

Stable oxygen radicals can be substantially stable. Substantially stable can mean that the stable oxygen radical can remain at a concentration greater than about 75% relative to the concentration on day 1, greater than about 80%, greater than about 85%, greater than about 90%, greater than about 95%, greater than about 96%, greater than about 97%, greater than about 98%, or greater than about 99% over a given time period as described above. For example, in one embodiment, the stable oxygen is at a concentration greater than about 95% relative to day 1 for at least 1 year. In another embodiment, the at least one oxygen radical is at a concentration greater than about 98% for at least 1 year.

Stable can mean that, after 1 year, the oxygen radical can remain at a concentration greater than 75% relative to the concentration on day 1 or the day is was produced, greater than 80% relative to the concentration on day 1 or the day is was produced, greater than 85% relative to the concentration on day 1 or the day is was produced, greater than 90% relative to the concentration on day 1 or the day is was produced, greater than 95% relative to the concentration on day 1 or the day is was produced, greater than 96% relative to the concentration on day 1 or the day is was produced, greater than 97% relative to the concentration on day 1 or the day is was produced, greater than 98% relative to the concentration on day 1 or the day is was produced, or greater than 99% relative to the concentration on day 1 or the day is was produced. For example, in one embodiment, the stable oxygen is at a concentration greater than about 95% relative to day 1 for at least 1 year. In another embodiment, the at least one oxygen radical is at a concentration greater than about 98% for at least 1 year.

Stable can mean that, after 5 years, the oxygen radical can remain at a concentration greater than 75% relative to the concentration on day 1 or the day is was produced, greater than 80% relative to the concentration on day 1 or the day is was produced, greater than 85% relative to the concentration on day 1 or the day is was produced, greater than 90% relative to the concentration on day 1 or the day is was produced, greater than 95% relative to the concentration on day 1 or the day is was produced, greater than 96% relative to the concentration on day 1 or the day is was produced, greater than 97% relative to the concentration on day 1 or the day is was produced, greater than 98% relative to the concentration on day 1 or the day is was produced, or greater than 99% relative to the concentration on day 1 or the day is was produced. For example, in one embodiment, the stable oxygen is at a concentration greater than about 95% relative to day 1 for at least 1 year. In another embodiment, the at least one oxygen radical is at a concentration greater than about 98% for at least 1 year.

Stable can mean that, after 10 years, the oxygen radical can remain at a concentration greater than 75% relative to the concentration on day 1 or the day is was produced, greater than 80% relative to the concentration on day 1 or the day is was produced, greater than 85% relative to the concentration on day 1 or the day is was produced, greater than 90% relative to the concentration on day 1 or the day is was produced, greater than 95% relative to the concentration on day 1 or the day is was produced, greater than 96% relative to the concentration on day 1 or the day is was produced, greater than 97% relative to the concentration on day 1 or the day is was produced, greater than 98% relative to the concentration on day 1 or the day is was produced, or greater than 99% relative to the concentration on day 1 or the day is was produced. For example, in one embodiment, the stable oxygen is at a concentration greater than about 95% relative to day 1 for at least 1 year. In another embodiment, the at least one oxygen radical is at a concentration greater than about 98% for at least 1 year.

Stability as used herein can also refer to the amount of a particular specie when compared to a reference sample. In some embodiments, the reference sample can be made in 1 L vessels with 0.9% isotonic solution electrolyzed with 3 Amps at 40° F., for 3 mins. In another embodiment, the reference sample can be made according to a process as otherwise described herein. The reference standard can also be bottle directly off the processing line as a “fresh” sample.

In other embodiments, the at least one oxygen radical is greater than about 86% stable for at least 4 years, greater than about 79% stable for at least 6 years, greater than about 72% stable for at least 8 years, greater than about 65% stable for at least 10 years, or 100% stable for at least 20 years such that stable can mean present and/or detectable.

In still other embodiments, the at least one oxygen radical is greater than about 95% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 96% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 97% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 98% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 99% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is 100% stable for at least 2 years, at least 3 years, at least 4 years, at least 5 years, at least 6 years, at least 7 years, at least 8 years, at least 9 years, at least 10 years, at least 15 years, or at least 20 years such that stable can mean present and/or detectable.

The stability of oxygen radicals can also be stated as a decay rate over time. Substantially stable can mean a decay rate less than about 1% per month, less than about 2% per month, less than about 3% per month, less than about 4% per month, less than about 5% per month, less than about 6% per month, less than about 10% per month, less than about 3% per year, less than about 4% per year, less than about 5% per year, less than about 6% per year, less than about 7% per year, less than about 8% per year, less than about 9% per year, less than about 10% per year, less than about 15% per year, less than about 20% per year, less than about 25% per year, between less than about 3% per month and less than about 7% per year.

In other embodiments, stability can be expressed as a half-life. A half-life of the stable oxygen radical can be about 6 months, about 1 year, about 2 years, about 3 years, about 4 years, about 5 years, about 10 years, about 15 years, about 20 years, about 24 years, about 30 years, about 40 years, about 50 years, greater than about 1 year, greater than about 2 years, greater than about 10 years, greater than about 20 years, greater than about 24 years, between about 1 year and about 30 years, between about 6 years and about 24 years, or between about 12 years and about 30 years.

Reactive species' concentrations in the life enhancing solutions, detected by fluorescence photo spectroscopy, may not significantly decrease in time. Mathematical models show that bound HOCl—*O2- complexes are possible at room temperature. Molecular complexes can preserve volatile components of reactive species. For example, reactive species concentrations in whole blood as a result of molecular complexes may prevent reactive species degradation over time.

Reactive species can be further divided into “reduced species” (RS) and “reactive oxygen species” (ROS). Reactive species can be formed from water molecules and sodium chloride ions when restructured through a process of forced electron donation. Electrons from lower molecular energy configurations in the salinated water may be forced into higher, more reactive molecular configurations. The species from which the electron was taken can be “electron hungry” and is called the RS and can readily become an electron acceptor (or proton donor) under the right conditions. The species that obtains the high-energy electron can be an electron donor and is called the ROS and may energetically release these electrons under the right conditions.

When an energetic electron in ROS is unpaired it is called a “radical”. ROS and RS can recombine to neutralize each other by the use of a catalytic enzyme. Three elements, (1) enzymes, (2) electron acceptors, and (3) electron donors can all be present at the same time and location for neutralization to occur.

Depending on the parameters used to produce the composition, different components can be present at different concentrations. In one embodiment, the composition can include about 0.1 ppt, about 0.5 ppt, about 1 ppt, about 1.5 ppt, about 2 ppt, about 2.5 ppt, about 3 ppt, about 3.5 ppt, about 4 ppt, about 4.5 ppt, about 5 ppt, about 6 ppt, about 7 ppt, about 8 ppt, about 9 ppt, about 10 ppt, about 20 ppt, about 50 ppt, about 100 ppt, about 200 ppt, about 400 ppt, about 1,000 ppt, between about 0.1 ppt and about 1,000 ppt, between about 0.1 ppt and about 100 ppt, between about 0.1 ppt and about 10 ppt, between about 2 ppt and about 4 ppt, at least about 0.1 ppt, at least about 2 ppt, at least about 3 ppt, at most about 10 ppt, or at most about 100 ppt of OCl—. In some embodiments, OCl— can be present at about 3 ppt. In other embodiments, OCl— can be the predominant chlorine containing species in the composition.

In order to determine the relative concentrations and rates of production of each of these during electrolysis, certain general chemical principles can be helpful:

1) A certain amount of Gibbs free energy is required for construction of the molecules; Gibbs free energy is proportional to the differences in electrode potentials listed in FIG. 2. Reactions with large energy requirements are less likely to happen, for example an electrode potential of −2.71V (compared to Hydrogen reduction at 0.00V) is required to make sodium metal:

Na⁺+1e⁻→Na₍ₛ₎

Such a large energy difference requirement makes this reaction less likely to happen compared to other reactions with smaller energy requirements. Electron(s) from the electrodes may be preferentially used in the reactions that require lesser amounts of energy, such as the production of hydrogen gas.

2) Electrons and reactants are required to be at the same micro-locality on the electrodes. Reactions that require several reactants may be less likely to happen, for example:

Cl₂+6H₂O→10e⁻+2ClO³⁻+12H⁺

requires that 6 water molecules and a Cl2 molecule to be at the electrode at the same point at the same time and a release of 10 electrons to simultaneously occur. The probability of this happening generally is smaller than other reactions requiring fewer and more concentrated reactants to coincide, but such a reaction may still occur.

3) Reactants generated in preceding generations can be transported or diffuse to the electrode where reactions happen. For example, dissolved oxygen (O2) produced on the anode from the first generation can be transported to the cathode in order to produce superoxides and hydrogen peroxide in the second generation. Ions can be more readily transported: they can be pulled along by the electric field due to their electric charge. In order for chlorates, to be generated, for example, HClO2 can first be produced to start the cascade, restrictions for HClO2 production can also restrict any subsequent chlorate production. Lower temperatures can prevent HClO2 production.

Stability and concentration of the above products can depend, in some cases substantially, on the surrounding environment. The formation of complexes and water clusters can affect the lifetime of the moieties, especially the free radicals.

In a pH-neutral aqueous solution (pH around 7.0) at room temperature, superoxide free radicals (O2*-) have a half-life of 10's of milliseconds and dissolved ozone (O3) has a half-life of about 20 min. Hydrogen peroxide (H2O2) is relatively long-lived in neutral aqueous environments, but this can depend on redox potentials and UV light. Other entities such as HCl and NaOH rely on acidic or basic environments, respectively, in order to survive. In pH-neutral solutions, H+ and OH— ions have concentrations of approximately 1 part in 10,000,000 in the bulk aqueous solution away from the electrodes. H— and 1O can react quickly. The stability of most of these moieties mentioned above can depend on their microenvironment.

Superoxides and ozone can form stable Van de Waals molecular complexes with hypochlorites. Clustering of polarized water clusters around charged ions can also have the effect of preserving hypochlorite-superoxide and hypochlorite-ozone complexes. Such complexes can be built through electrolysis on the molecular level on catalytic substrates, and may not occur spontaneously by mixing together components. Hypochlorites can also be produced spontaneously by the reaction of dissolved chlorine gas (Cl2) and water. As such, in a neutral saline solution the formation of on or more of the stable molecules and complexes may exist: dissolved gases: O2, H2, Cl2; hypochlorites: OCl—, HOCl, NaOCl; hypochlorates: HClO2, ClO2, HClO3, HClO4; hydrogen peroxide: H2O2; ions: Na+, Cl—, H+, H—, OH—; ozone: O3, O4*-; singlet oxygen: 1O; hydroxyl free radical: OH*—; superoxide complexes: HOCl—O2*-; and ozone complexes: HOCl—O3. One or more of the above molecules can be found within the compositions and composition described herein.

A complete quantum chemical theory can be helpful because production is complicated by the fact that different temperatures, electrode geometries, flows and ion transport mechanisms and electrical current modulations can materially change the relative/absolute concentrations of these components, which could result in producing different distinct compositions. As such, the selection of production parameters can be critical. The amount of time it would take to check all the variations experimentally may be prohibitive.

The amount of chlorine can be predetermined by adjusting the amount of salt used, the temperature maintained during the electrochemical or electrolysis process, the time spent for processing and other parameters. chlorine and/or salts thereof can be present in an amount of from 1 to 1000 ppm. Preferably, chlorine can be present in an amount of from 1 to 500 ppm. More preferably chlorine can be present in an amount of from 1 to 100 ppm. Chlorine can be present for example in an amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 ppm.

The chlorine concentration of the electrolyzed solution can also be in ranges and can be about 5 ppm, about 10 ppm, about 15 ppm, about 20 ppm, about 21 ppm, about 22 ppm, about 23 ppm, about 24 ppm, about 25 ppm, about 26 ppm, about 27 ppm, about 28 ppm, about 29 ppm, about 30 ppm, about 31 ppm, about 32 ppm, about 33 ppm, about 34 ppm, about 35 ppm, about 36 ppm, about 37 ppm, about 38 ppm, less than about 38 ppm, less than about 35 ppm, less than about 32 ppm, less than about 28 ppm, less than about 24 ppm, less than about 20 ppm, less than about 16 ppm, less than about 12 ppm, less than about 5 ppm, between about 30 ppm and about 34 ppm, between about 28 ppm and about 36 ppm, between about 26 ppm and about 38 ppm, between about 20 ppm and about 38 ppm, between about 5 ppm and about 34 ppm, between about 10 ppm and about 34 ppm, or between about 15 ppm and about 34 ppm. In one embodiment, the chlorine concentration is about 32 ppm. In another embodiment, the chlorine concentration is less than about 41 ppm.

The saline concentration in the electrolyzed solution can be about 0.10% w/v, about 0.11% w/v, about 0.12% w/v, about 0.13% w/v, about 0.14% w/v, about 0.15% w/v, about 0.16% w/v, about 0.17% w/v, about 0.18% w/v, about 0.19% w/v, about 0.20% w/v, about 0.30% w/v, about 0.40% w/v, about 0.50% w/v, about 0.60% w/v, about 0.70% w/v, between about 0.10% w/v and about 0.20% w/v, between about 0.11% w/v and about 0.19% w/v, between about 0.12% w/v and about 0.18% w/v, between about 0.13% w/v and about 0.17% w/v, or between about 0.14% w/v and about 0.16% w/v.

The saline concentration in the electrolyzed solution can be at least 0.10% w/v, at least 0.11% w/v, at least 0.12% w/v, at least 0.13% w/v, at least 0.14% w/v, at least 0.15% w/v, at least 0.16% w/v, at least 0.17% w/v, at least 0.18% w/v, at least 0.19% w/v, at least 0.20% w/v, at least 0.30% w/v, at least 0.40% w/v, at least 0.50% w/v, at least 0.60% w/v, at least 0.70% w/v, between 0.10% w/v and 0.20% w/v, between 0.11% w/v and 0.19% w/v, between 0.12% w/v and 0.18% w/v, between 0.13% w/v and 0.17% w/v, or between 0.14% w/v and 0.16% w/v.

The composition generally can include electrolytic and/or catalytic products of pure saline that mimic redox signaling molecular compositions of the native salt water compounds found in and around human cells. The composition can be fine-tuned to mimic or mirror molecular compositions of different biological media. For example, ROS such as those occurring or being produced naturally in the mitochondria include 02- and hydrogen peroxide (Zhang et al. Mitochondrial reactive oxygen species-mediated signaling in endothelial cells. Heart May 2007 vol. 292 no. 5 H2023-H2031 which is incorporated herein in its entirety by reference.). The composition can have reactive species other than chlorine present. As described, species present in the compositions and compositions described herein can include, but are not limited to O2, H2, Cl2, OCl—, HOC, NaOCl, HClO2, ClO2, HClO3, HClO4, H2O2, Na+, Cl—, H+, H—, OH—, O3, O4*-, 1O, OH*—, HOCl—O2*-, HOCl—O3, O2*, HO2*, NaCl, HCl, NaOH, and water clusters: n*H2O— induced dipolar layers around ions, several variations.

In some embodiments, substantially no organic material is present in the compositions described. Substantially no organic material can be less than about 0.1 ppt, less than about 0.01 ppt, less than about 0.001 ppt or less than about 0.0001 ppt of total organic material.

The composition can be stored and bottled as needed to ship to consumers. The composition can have a shelf life of about 5 days, about 30 days, about 3 months, about 6 months, about 9 months, about 1 year, about 1.5 years, about 2 years, about 3 years, about 5 years, about 10 years, at least about 5 days, at least about 30 days, at least about 3 months, at least about 6 months, at least about 9 months, at least about 1 year, at least about 1.5 years, at least about 2 years, at least about 3 years, at least about 5 years, at least about 10 years, between about 5 days and about 1 year, between about 5 days and about 2 years, between about 1 year and about 5 years, between about 90 days and about 3 years, between about 90 days and about 5 year, or between about 1 year and about 3 years.

Quality Assurance testing can be done on every batch before the batch can be approved for bottling or can be performed during or after bottling. A 16 oz. sample bottle can be taken from each complete batch and analyzed. Determinations for presence of contaminants such as heavy metals or chlorates can be performed. Then pH, Free and Total Chlorine concentrations and reactive molecule concentrations of the active ingredients can be analyzed by fluorospectroscopy methods. These results can be compared to those of a standard solution which is also tested along side every sample. If the results for the batch fall within a certain range relative to the standard solution, it can be approved. A chemical chromospectroscopic MS analysis can also be run on random samples to determine if contaminants from the production process are present.

The composition can be consumed by ingestion. In other embodiments, the composition can be provided as a solution for injection. In some embodiments, injection can be subcutaneous, intra-luminal, site specific, or intramuscular. Intravenous injection can also be desirable. Most preferably, the composition is used topically. The composition can be packaged in plastic medical solution pouches having volumes of about 4 oz, about 8 oz, about 16 oz, about 32 oz, about 48 oz, about 64 oz, about 80 oz, about 96 oz, about 112 oz, about 128 oz, about 144 oz, about 160 oz, or any range created using any of these values, and these pouches can be used with common intravenous administration systems.

When administered, it can be administered once, twice, three times, four times or more a day. Each administration can be about 1 oz, about 2 oz, about 3 oz, about 4 oz, about 5 oz, about 6 oz, about 7 oz, about 8 oz, about 9 oz, about 10 oz, about 11 oz, about 12 oz, about 16 oz, about 20 oz, about 24 oz, about 28 oz, about 32 oz, about 34 oz, about 36 oz, about 38 oz, about 40 oz, about 46 oz, between about 1 oz and about 32 oz, between about 1 oz and about 16 oz, between about 1 oz and about 8 oz, at least about 2 oz, at least about 4 oz, or at least about 8 oz. In one embodiment, the composition can be administered at a rate of about 4 oz twice a day.

In other embodiments, the administration can be acute or long term. For example, the composition can be administered for a day, a week, a month, a year or longer. In other embodiments, the composition can simply be taken as needed.

Compositions of the invention can be formulated into any suitable aspect, such as, for example, aerosols, liquids, elixirs, syrups, tinctures and the like.

When administered as a liquid composition, it can be administered once, twice, three times, four times or more a day. Each administration can be about 1 oz, about 2 oz, about 3 oz, about 4 oz, about 5 oz, about 6 oz, about 7 oz, about 8 oz, about 9 oz, about 10 oz, about 11 oz, about 12 oz, about 16 oz, about 20 oz, about 24 oz, about 28 oz, about 32 oz, about 34 oz, about 36 oz, about 38 oz, about 40 oz, about 46 oz, between about 1 oz and about 32 oz, between about 1 oz and about 16 oz, between about 1 oz and about 8 oz, at least about 2 oz, at least about 4 oz, or at least about 8 oz. In one embodiment, the composition can be administered at a rate of about 4 oz twice a day.

In other embodiments, the administration can be acute or long term. For example, the composition can be administered for a day, a week, a month, a year or longer.

In a physiological aspect, a decrease in the rate of muscle glycogen depletion is proportional to an increase in endurance. To that end, the change in muscle glycogen depletion is a benchmark for physical endurance. The greater the decrease in muscle glycogen depletion, the greater the physical endurance. Given the beneficial impact of RXNs on the health and wellbeing of individuals who take it, this 30% decrease in muscle glycogen depletion is a benchmark itself. In one embodiment, a decrease in muscle glycogen depletion following exercise correlates with an increase in athletic endurance. In another embodiment, at least a 1% decrease in muscle glycogen depletion is indicative of an increase in athletic endurance. In another embodiment, at least a 5% decrease in muscle glycogen depletion is indicative of an increase in athletic endurance. In another embodiment, at least a 10% decrease in muscle glycogen depletion is indicative of an increase in athletic endurance. In another embodiment, at least a 15% decrease in muscle glycogen depletion is indicative of an increase in athletic endurance. In another embodiment, at least a 20% decrease in muscle glycogen depletion is indicative of an increase in athletic endurance. In another embodiment, at least a 25% decrease in muscle glycogen depletion is indicative of an increase in athletic endurance. In other embodiments, there is at least a 1% decrease, a 2% decrease, a 3% decrease, a 4% decrease, a 5% decrease, a 6% decrease, a 7% decrease, a 8% decrease, a 9% decrease, a 10% decrease, a 11% decrease, a 12% decrease, a 13% decrease, a 14% decrease, a 15% decrease, a 16% decrease, a 17% decrease, a 18% decrease, a 19% decrease, a 20% decrease, a 21% decrease, a 22% decrease, a 23% decrease, a 24% decrease, a 25% decrease, a 26% decrease, a 27% decrease, a 28% decrease, a 29% decrease, a 30% decrease, a 31% decrease, a 32% decrease, a 33% decrease, a 34% decrease, a 35% decrease, a 36% decrease, a 37% decrease, a 38% decrease, a 39% decrease, or at least a 40% decrease in the rate of muscle glycogen depletion.

### Example 1

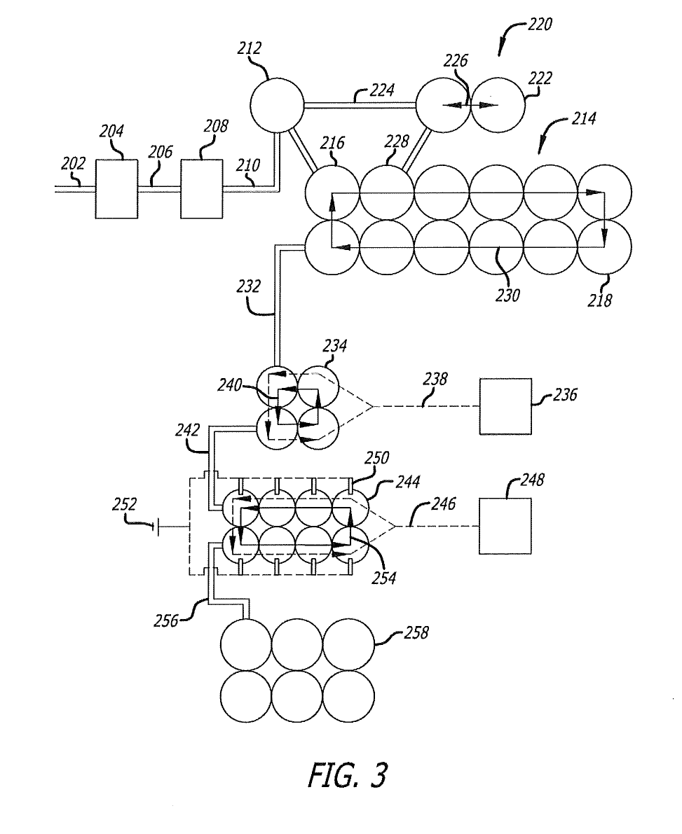

FIG. 3 illustrates a plan view of a process and system for producing a composition according to the present description. One skilled in the art understands that changes can be made to the system to alter the composition, and these changes are within the scope of the present description.

Incoming water 202 can be subjected to reverse osmosis system 204 at a temperature of about 15-20° C. to achieve purified water 206 with about 8 ppm of total dissolved solids. Purified water 206, is then fed at a temperature of about 15-20° C. into distiller 208 and processed to achieve distilled water 210 with about 0.5 ppm of total dissolved solids. Distilled water 210 can then be stored in tank 212.

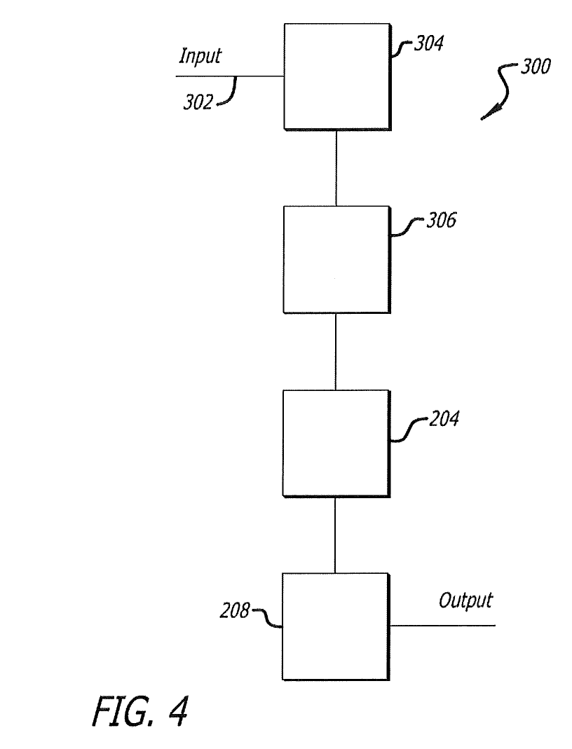

FIG. 4 illustrates an example system for preparing water for further processing into a therapeutic composition. System 300 can include a water source 302 which can feed directly into a carbon filter 304. After oils, alcohols, and other volatile chemical residuals and particulates are removed by carbon filter 304, the water can be directed to resin beds within a water softener 306 which can remove dissolved minerals. Then, as described above, the water can pass through reverse osmosis system 204 and distiller 208.

Referring again to FIG. 3, distilled water 210 can be gravity fed as needed from tank 212 into saline storage tank cluster 214 using line 216. Saline storage tank cluster 214 in one embodiment can include twelve tanks 218. Each tank 218 can be filled to about 1,300 gallons with distilled water 210. A handheld meter can be used to test distilled water 210 for salinity.

Saline storage tank cluster 214 is then salted using a brine system 220. Brine system 220 can include two brine tanks 222. Each tank can have a capacity of about 500 gallons. Brine tanks 222 are filled to 475 gallons with distilled water 210 using line 224 and then NaCl is added to the brine tanks 222 at a ratio of about 537.5 g/gal of liquid. At this point, the water is circulated 226 in the brine tanks 222 at a rate of about 2,000 gal/hr for about 4 days.

Prior to addition of brine to tanks 218, the salinity of the water in tanks 218 can be tested using a handheld conductivity meter such as an YSI ECOSENSE® ecp300 (YSI Inc., Yellow Springs, Ohio). Any corrections based on the salinity measurements can be made at this point. Brine solution 228 is then added to tanks 218 to achieve a salt concentration of about 10.75 g/gal. The salted water is circulated 230 in tanks 218 at a rate of about 2,000 gal/hr for no less than about 72 hours. This circulation is performed at room temperature. A handheld probe can again be used to test salinity of the salinated solution. In one embodiment, the salinity is about 2.8 ppth.

In one method for filling and mixing the salt water in the brine holding tanks, the amount of liquid remaining in the tanks is measured. The amount of liquid remaining in a tank is measured by recording the height that the liquid level is from the floor that sustains the tank, in centimeters, and referencing the number of gallons this height represents. This can be done from the outside of the tank if the tank is semi-transparent. The initial liquid height in both tied tanks can also be measured. Then, after ensuring that the output valve is closed, distilled water can be pumped in. The amount of distilled water that is being pumped into a holding tank can then be calculated by measuring the rise in liquid level: subtracting the initial height from the filled height and then multiplying this difference by a known factor.

The amount of salt to be added to the tank is then calculated by multiplying 11 grams of salt for every gallon of distilled water that has been added to the tank. The salt can be carefully weighed out and dumped into the tank.

The tank is then agitated by turning on the recirculation pump and then opening the top and bottom valves on the tank. Liquid is pumped from the bottom of the tank to the top. The tank can be agitated for three days before it may be ready to be processed.

After agitating the tank for more than 6 hours, the salinity is checked with a salinity meter by taking a sample from the tank and testing it. Salt or water can be added to adjust the salinity within the tanks. If either more water or more salt is added then the tanks are agitated for 6 more hours and tested again. After about three days of agitation, the tank is ready to be processed.

Salinated water 232 is then transferred to cold saline tanks 234. In one embodiment, four 250 gal tanks are used. The amount of salinated water 232 moved is about 1,000 gal. A chiller 236 such as a 16 ton chiller is used to cool heat exchangers 238 to about 0-5° C. The salinated water is circulated 240 through the heat exchangers which are circulated with propylene glycol until the temperature of the salinated water is about 4.5-5.8° C. Chilling the 1,000 gal of salinated water generally takes about 6-8 hr.

Cold salinated water 242 is then transferred to processing tanks 244. In one embodiment, eight tanks are used and each can have a capacity of about 180 gal. Each processing tank 244 is filled to about 125 gal for a total of 1,000 gal. Heat exchangers 246 are again used to chill the cold salinated water 242 added to processing tanks 244. Each processing tank can include a cylinder of chilling tubes and propylene glycol can be circulated. The heat exchangers can be powered by a 4-5 ton chiller 248. The temperature of cold salinated water 242 can remain at 4.5-5.8° C. during processing.

Prior to transferring aged salt water to processing tanks, the aged salt water can be agitated for about 30 minutes to sufficiently mix the aged salt water. Then, the recirculation valves can then be closed, the appropriate inlet valve on the production tank is opened, and the tank filled so that the salt water covers the cooling coils and comes up to the fill mark (approximately 125 gallons).

Once the aged salt water has reached production temperature, the pump is turned off but the chiller left on. The tank should be adequately agitated or re-circulated during the whole duration of electrochemical processing and the temperature should remain constant throughout.

Each processing tank 244 includes electrode 250. Electrodes 250 can be 3 inches tall circular structures formed of titanium and plated with platinum. Electrochemical processing of the cold salinated water can be run for 8 hr. A power supply 252 is used to power the eight electrodes (one in each processing tank 244) to 7 amps each for a total of 56 amps. The cold salinated water is circulated 254 during electrochemical processing at a rate of about 1,000 gal/hr.

An independent current meter can be used to set the current to around 7.0 Amps. Attention can be paid to ensure that the voltage does not exceed 12V and does not go lower than 9V. Normal operation can be about 10V.

A run timer can be set for a prescribed time (about 4.5 to 5 hours). Each production tank can have its own timer and/or power supply. Electrodes should be turned off after the timer has expired.

The production tanks can be checked periodically. The temperature and/or electrical current can be kept substantially constant. At the beginning, the electrodes can be visible from the top, emitting visible bubbles. After about 3 hours, small bubbles of un-dissolved oxygen can start building up in the tank as oxygen saturation occurs, obscuring the view of the electrodes. A slight chlorine smell can be normal.

After the 8 hour electrochemical processing is complete, life enhancing water 256 has been created with a pH of about 6.8-8.2 and 32 ppm of chlorine. The composition 256 is transferred to storage tanks 258. The product ASEA can be made by this process. Preferably, the product ASEA is made by the process of this Example 1.

### Example 2

### Characterization of a Solution Produced as Described in Example 1

A composition produced as described in Example 1 was analyzed using a variety of different characterization techniques. ICP/MS and 35Cl NMR were used to analyze and quantify chlorine content. Headspace mass spectrometry analysis was used to analyze adsorbed gas content in the composition. 1H NMR was used to verify the organic matter content in the composition. 31P NMR and EPR experiments utilizing spin trap molecules were used to explore the composition for free radicals.

The composition was received and stored at about 4° C. when not being used.

Chlorine NMR

Sodium hypochlorite solutions were prepared at different pH values. 5% sodium hypochlorite solution had a pH of 12.48. Concentrated nitric acid was added to 5% sodium hypochlorite solution to create solutions that were at pH of 9.99, 6.99, 5.32, and 3.28. These solutions were then analyzed by NMR spectroscopy. The composition had a measured pH of 8.01 and was analyzed directly by NMR with no dilutions.

NMR spectroscopy experiments were performed using a 400 MHz Bruker spectrometer equipped with a BBO probe. 35Cl NMR experiments were performed at a frequency of 39.2 MHz using single pulse experiments. A recycle delay of 10 seconds was used, and 128 scans were acquired per sample. A solution of NaCl in water was used as an external chemical shift reference. All experiments were performed at room temperature.

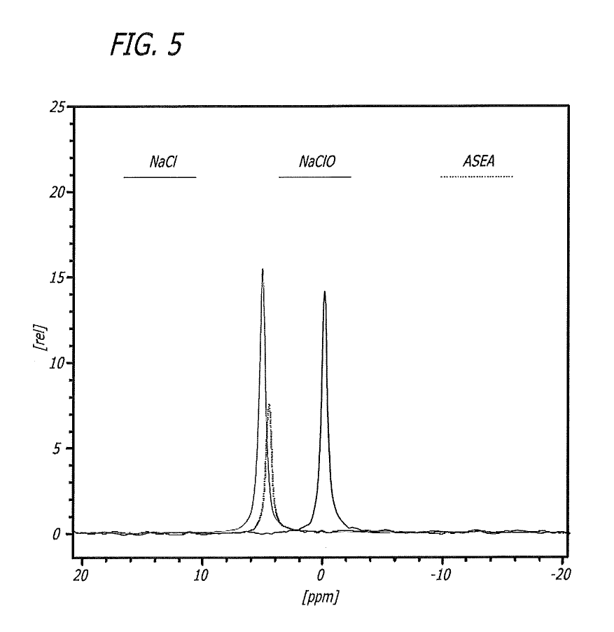

35Cl NMR spectra were collected for NaCl solution, NaClO solutions adjusted to different pH values, and the composition. FIG. 5 illustrates a Cl35 spectrum of NaCl, NaClO solution at a pH of 12.48, and the composition. The chemical shift scale was referenced by setting the Cl— peak to 0 ppm. NaClO solutions above a pH=7 had identical spectra with a peak at approximately 5.1 ppm. Below pH of 7.0, the ClO— peak disappeared and was replaced by much broader, less easily identifiable peaks. The composition was presented with one peak at approximately 4.7 ppm, from ClO— in the composition. This peak was integrated to estimate the concentration of ClO— in the composition, which was determined to be 2.99 ppt or 0.17 M of ClO— in the composition.

Proton NMR

An ASEA sample was prepared by adding 550 μL of ASEA and 50 μL of D20 (Cambridge Isotope Laboratories) to an NMR tube and vortexing the sample for 10 seconds. 1H NMR experiments were performed on a 700 MHz Bruker spectrometer equipped with a QNP cryogenically cooled probe. Experiments used a single pulse with pre-saturation on the water resonance experiment. A total of 1024 scans were taken. All experiments were performed at room temperature.

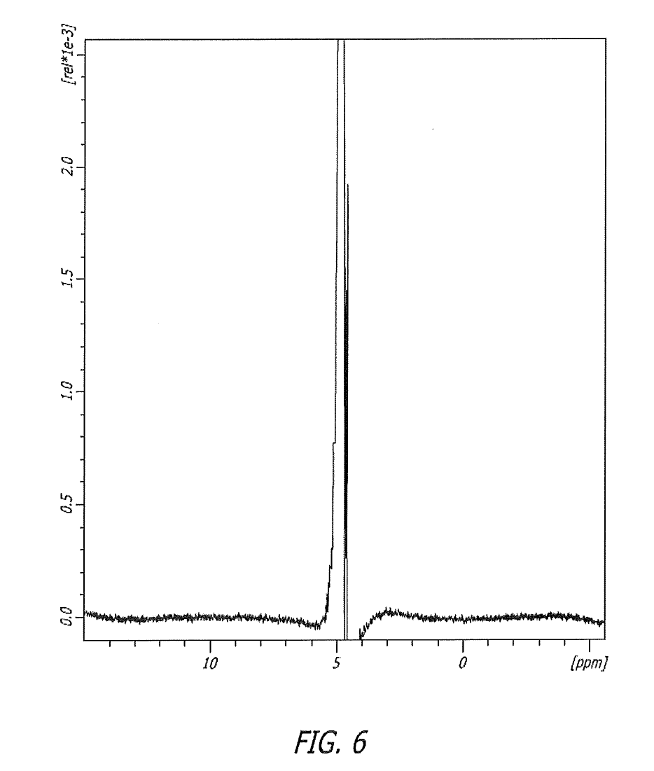

A 1H NMR spectrum of the composition was determined and is presented in FIG. 6. Only peaks associated with water were able to be distinguished from this spectrum. This spectrum show that very little if any organic material can be detected in the composition using this method.

Phosphorous NMR and Mass Spectrometry

DIPPMPO (5-(Diisopropoxyphosphoryl)-5-1-pyrroline-N-oxide) (VWR) samples were prepared by measuring about 5 mg of DIPPMPO into a 2 mL centrifuge tube. This tube then had 550 μL of either the composition or water added to it, followed by 50 μL of D2O. A solution was also prepared with the composition but without DIPPMPO. These solutions were vortexed and transferred to NMR tubes for analysis. Samples for mass spectrometry analysis were prepared by dissolving about 5 mg of DIPPMPO in 600 μL of the composition and vortexing, then diluting the sample by adding 100 μL of sample and 900 μL of water to a vial and vortexing.

NMR experiments were performed using a 700 MHz Bruker spectrometer equipped with a QNP cryogenically cooled probe. Experiments performed were a single 30° pulse at a 31P frequency of 283.4 MHz. A recycle delay of 2.5 seconds and 16384 scans were used. Phosphoric acid was used as an external standard. All experiments were performed at room temperature.

Mass spectrometry experiments were performed by directly injecting the ASEA/DIPPMPO sample into a Waters/Synapt Time of Flight mass spectrometer. The sample was directly injected into the mass spectrometer, bypassing the LC, and monitored in both positive and negative ion mode.

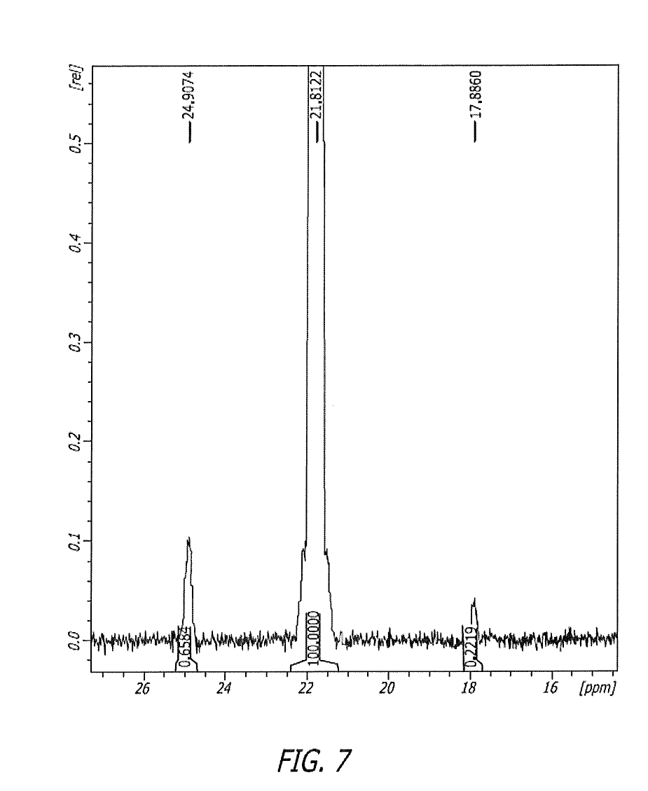

31P NMR spectra were collected for DIPPMPO in water, the composition alone, and the composition with DIPPMPO added to it. An external reference of phosphoric acid was used as a chemical shift reference. FIG. 7 illustrates a 31P NMR spectrum of DIPPMPO combined with the composition. The peak at 21.8 ppm was determined to be DIPPMPO and is seen in both the spectrum of DIPPMPO with the composition (FIG. 7) and without the composition (not pictured). The peak at 24.9 ppm is most probably DIPPMPO/OH. as determined in other DIPPMPO studies. This peak may be seen in DIPPMPO mixtures both with and without the composition, but is detected at a much greater concentration in the solution with the composition. In the DIPPMPO mixture with the composition, there is another peak at 17.9 ppm. This peak may be from another radical species in the composition such as OOH. or possibly a different radical complex. The approximate concentrations of spin trap complexes in the composition/DIPPMPO solution are as follows:

| Solution | Concentration | |
|---|---|---|
| DIPPMPO | 36.6 | mM |
| DIPPMPO/OH• | 241 | μM |
| DIPPMPO/radical | 94 | μM |

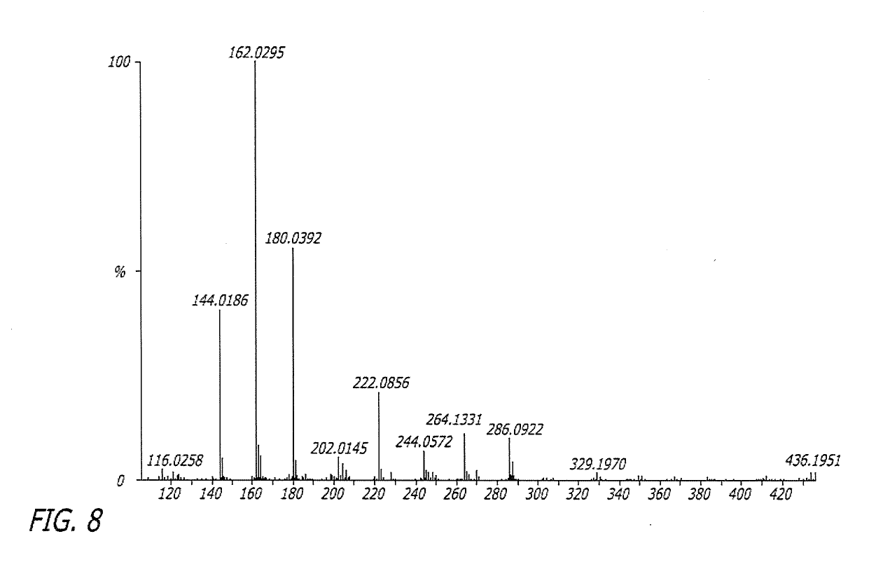

Mass spectral data was collected in an attempt to determine the composition of the unidentified radical species. The mass spectrum shows a parent peak and fragmentation pattern for DIPPMPO with m/z peaks at 264, 222, and 180, as seen in FIG. 8. FIG. 8 also shows peaks for the DIPPMPO/Na adduct and subsequent fragments at 286, 244, and 202 m/z. Finally, FIG. 8 demonstrates peaks for one DIPPMPO/radical complex with m/z of 329. The negative ion mode mass spectrum also had a corresponding peak at m/z of 327. There are additional peaks at 349, 367, and 302 at a lower intensity as presented in FIG. 8. None of these peaks could be positively confirmed. However, there are possible structures that would result in these mass patterns. One possibility for the peak generated at 329 could be a structure formed from a radical combining with DIPPMPO. Possibilities of this radical species include a nitroxyl-peroxide radical (HNO—HOO.) that may have formed in the composition as a result of reaction with nitrogen from the air. Another peak at 349 could also be a result of a DIPPMPO/radical combination. Here, a possibility for the radical may be hypochlorite-peroxide (HOCl—HOO.). However, the small intensity of this peak and small intensity of the corresponding peak of 347 in the negative ion mode mass spectrum indicate this could be a very low concentration impurity and not a compound present in the ASEA composition.

ICP/MS Analysis

Samples were analyzed on an Agilent 7500 series inductively-coupled plasma mass spectrometer (ICP-MS) in order to confirm the hypochlorite concentration that was determined by NMR. A stock solution of 5% sodium hypochlorite was used to prepare a series of dilutions consisting of 300 ppb, 150 ppb, 75 ppb, 37.5 ppb, 18.75 ppb, 9.375 ppb, 4.6875 ppb, 2.34375 ppb, and 1.171875 ppb in deionized Milli-Q water. These standards were used to establish a standard curve.

Based on NMR hypochlorite concentration data, a series of dilutions was prepared consisting of 164.9835 ppb, 82.49175 ppb, 41.245875 ppb, 20.622937 ppb, 10.311468 ppb, and 5.155734 ppb. These theoretical values were then compared with the values determined by ICP-MS analysis. The instrument parameters were as follows:

| | |
|---|---|
| Elements analyzed | ³⁵Cl, ³⁷Cl |
| # of points per mass | 20 |
| # of repetitions | 5 |
| Total acquisition time | 68.8 s |
| Uptake speed | 0.50 rps |
| Uptake time | 33 s |
| Stabilization time | 40 s |
| Tune | No Gas |
| Nebulizer flow rate | 1 mL/min |
| Torch power | 1500 W |

The results of the ICP-MS analysis are as follows:

| | Measured Concentration | Concentration by NMR |
|---|---|---|
| Dilution | (ppb) | (ppb) |
| 1 | 81 | 82 |
| 2 | 28 | 41 |
| 3 | 24 | 21 |
| 4 | 13 | 10 |
| 5 | 8 | 5 |

Dilutions were compared graphically to the ICP-MS signals and fit to a linear equation (R2=0.9522). Assuming linear behavior of the ICP-MS signal, the concentration of hypochlorite in the composition was measured to be 3.02 ppt. Concentration values were determined by calculating the concentration of dilutions of the initial composition and estimating the initial composition hypochlorite concentration to be 3 ppt (as determined from 35Cl NMR analysis). The ICP-MS data correlate well with the 35Cl NMR data, confirming a hypochlorite concentration of roughly ⅓% (3 ppt). It should be noted that ICP-MS analysis is capable of measuring total chlorine atom concentration in solution, but not specific chlorine species. The NMR data indicate that chlorine predominantly exists as ClO— in the composition.

Gas Phase Quadrupole MS

Sample Prep

Three sample groups were prepared in triplicate for the analysis: 1) Milli-Q deionized water 2) the composition, and 3) 5% sodium hypochlorite standard solution. The vials used were 20 mL headspace vials with magnetic crimp caps (GERSTEL). A small stir bar was placed in each vial (VWR) along with 10 mL of sample. The vials were capped, and then placed in a Branson model 5510 sonicator for one hour at 60° C.

The sonicator was set to degas which allowed for any dissolved gasses to be released from the sample into the headspace. After degassing, the samples were placed on a CTC PAL autosampler equipped with a heated agitator and headspace syringe. The agitator was set to 750 rpm and 95° C. and the syringe was set to 75° C. Each vial was placed in the agitator for 20 min prior to injection into the instrument. A headspace volume of 2.5 mL was collected from the vial and injected into the instrument.

Instrument Parameters

The instrument used was an Agilent 7890A GC system coupled to an Agilent 5975C El/Cl single quadrupole mass selective detector (MSD) set up for electron ionization. The GC oven was set to 40° C. with the front inlet and the transfer lines being set to 150° C. and 155° C. respectively. The carrier gas used was helium and it was set to a pressure of 15 PSI.

The MSD was set to single ion mode (SIM) in order to detect the following analytes:

| Analyte | Mass |
|---|---|
| Water | 18 |
| Nitrogen | 28 |
| Oxygen | 32 |
| Argon | 40 |
| Carbon Dioxide | 44 |
| Chlorine | 70 |
| Ozone | 48 |

The ionization source temperature was set to 230° C. and the quadrupole temperature was set to 150° C. The electron energy was set to 15 V.

Mass spectrometry data was obtained from analysis of the gas phase headspace of the water, the composition, and hypochlorite solution. The raw area counts obtained from the mass spectrometer were normalized to the area counts of nitrogen in order to eliminate any systematic instrument variation. Both nitrogen and water were used as standards because they were present in equal volumes in the vial with nitrogen occupying the headspace and water being the solvent. It was assumed that the overall volume of water and nitrogen would be the same for each sample after degassing. In order for this assumption to be correct, the ratio of nitrogen to water should be the same for each sample. A cutoff value for the percent relative standard deviation (% RSD) of 5% was used. Across all nine samples, a % RSD of 4.2 was observed. Of note, sample NaClO-3 appears to be an outlier, thus, when removed, the % RSD drops to 3.4%.

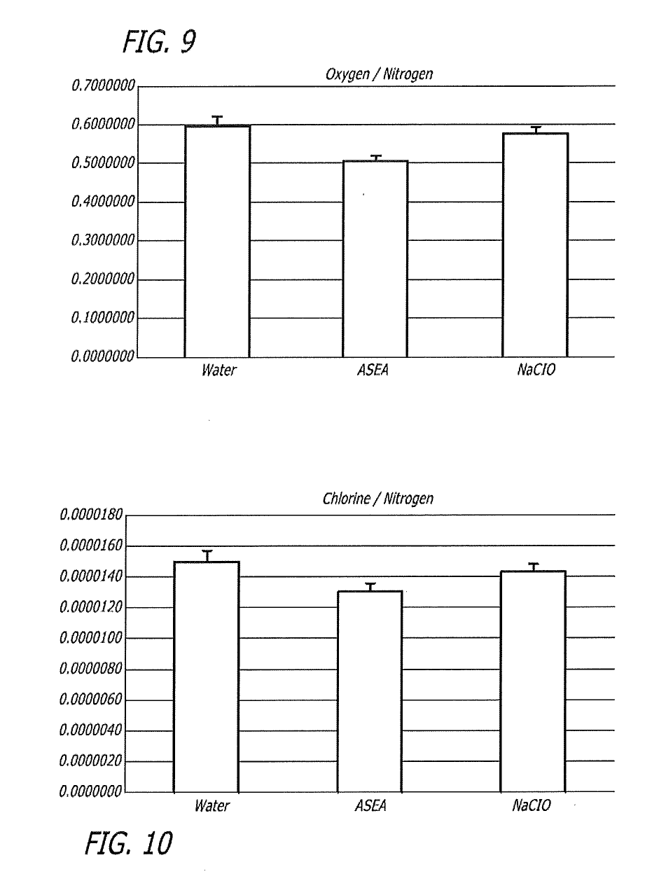

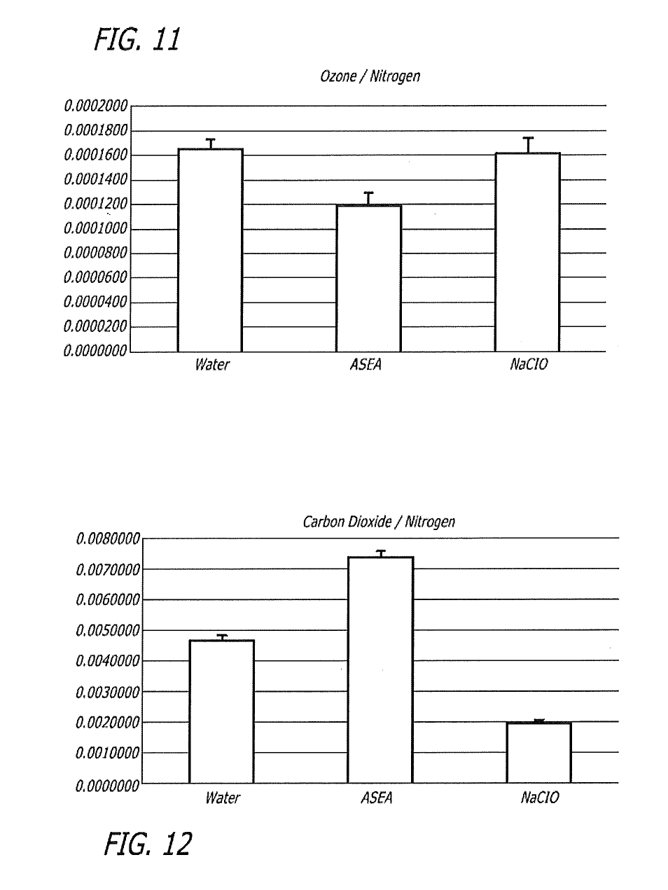

FIGS. 9-11 illustrate oxygen/nitrogen, chlorine/nitrogen, and ozone/nitrogen ratios. It appears that there were less of these gases released from the composition than from either water or nitrogen. It should be noted that the signals for both ozone and chlorine were very weak. Thus, there is a possibility that these signals may be due to instrument noise and not from the target analytes.

FIG. 12 illustrates the carbon dioxide to nitrogen ratio. It appears that there may have been more carbon dioxide released from the composition than oxygen. However, it is possible that this may be due to background contamination from the atmosphere.

Based on the above, more oxygen was released from both water and sodium hypochlorite than the composition.

EPR

Two different composition samples were prepared for EPR analysis. The composition with nothing added was one sample. The other sample was prepared by adding 31 mg of DIPPMPO to 20 mL of the composition (5.9 mM), vortexing, and placing the sample in a 4° C. refrigerator overnight. Both samples were placed in a small capillary tube which was then inserted into a normal 5 mm EPR tube for analysis.

EPR experiments were performed on a Bruker EMX 10/12 EPR spectrometer. EPR experiments were performed at 9.8 GHz with a centerfield position of 3500 Gauss and a sweepwidth of 100 Gauss. A 20 mW energy pulse was used with modulation frequency of 100 kHz and modulation amplitude of 1 G. Experiments used 100 scans. All experiments were performed at room temperature.

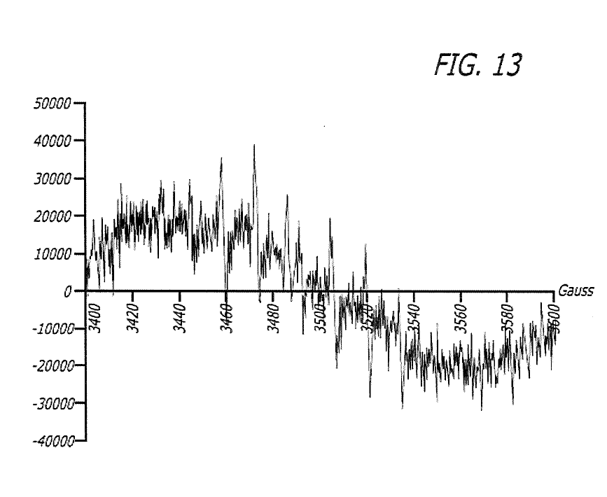

EPR analysis was performed on the composition with and without DIPPMPO mixed into the solution. FIG. 13 shows the EPR spectrum generated from DIPPMPO mixed with the composition. The composition alone showed no EPR signal after 100 scans (not presented). FIG. 13 illustrates an EPR splitting pattern for a free electron. This electron appears to be split by three different nuclei. The data indicate that this is a characteristic splitting pattern of OH. radical interacting with DMPO (similar to DIPPMPO). This pattern can be described by 14N splitting the peak into three equal peaks and 1H three bonds away splitting that pattern into two equal triplets. If these splittings are the same, it leads to a quartet splitting where the two middle peaks are twice as large as the outer peaks. This pattern may be seen in FIG. 13 twice, with the larger peaks at 3457 and 3471 for one quartet and 3504 and 3518 for the other quartet. In this case, the 14N splitting and the 1H splitting are both roughly 14G, similar to an OH* radical attaching to DMPO. The two quartet patterns in FIG. 13 are created by an additional splitting of 47G. This splitting is most likely from coupling to 31P, and similar patterns have been seen previously. The EPR spectrum in FIG. 13 indicates that there is a DIPPMPO/OH. radical species in the solution.

### Example 3

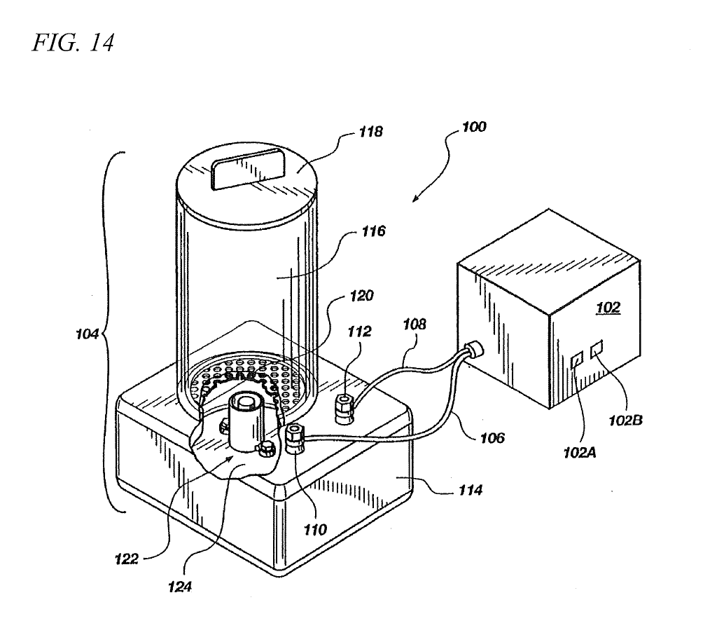

The electrolyzed fluid can be made in different types of vessels as long as the proper power sourced is used. One example of an apparatus that was used to make electrolyzed solution for treating fungal infections is that referred to in FIGS. 14-18.

Referring first to FIG. 14, which is a perspective view of a first presently preferred embodiment of the present invention generally represented at 100, includes a power supply 102 and a fluid receptacle represented at 104. The fluid receptacle 104 includes a base 114 upon which is attached a fluid vessel 116. The base 114 can preferably be fabricated from an insulative plastic material. The fluid vessel 116 is preferably fabricated from an inert clear plastic material which is compatible with biological processes as available in the art.

A lid 118 is provided to cover the fluid vessel 116 and keep contaminants out of the fluid vessel 116. A screen 120 is positioned to prevent foreign objects, which might accidentally fall into the fluid vessel 116, from falling to the bottom of the fluid vessel 116. The saline solution which is to be treated is placed into the fluid vessel 116, and the lid 118 placed, for the necessary period of time after which the electrolyzed saline solution can be withdrawn from the fluid vessel 116, for example into a syringe, for use. The fluid vessel 116 is sealed at its bottom by a floor 124 which is attached to the interior of the base 114.

An electrode assembly, generally represented at 122, is attached to the floor 124 so that any fluid in the fluid vessel is exposed to the electrode assembly 122. The electrode assembly 122 is electrically connected to the power supply 102 via terminals 110 and 112 and cables 106 and 108, respectively. The power supply 102 should deliver a controlled voltage and current to the electrode assembly 122 when fluid is placed into the fluid vessel 116. The voltage and current applied to the electrode assembly 122 will vary according to the fluid being electrolyzed. A control for setting and measuring the voltage 102A and a control for setting and measuring the current 102B is provided in the power supply. In accordance with the present invention, a low voltage of less than about 30 volts DC is used. Exemplary voltage and current values, and the advantages which accrue when using the preferred voltage and current values, will be explained shortly.

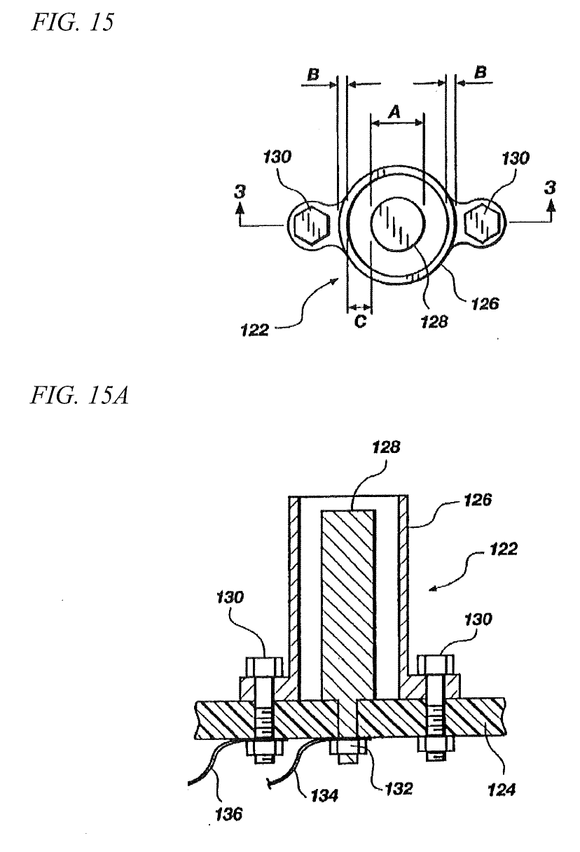

FIG. 15 is a top view of the electrode assembly 122 represented in FIG. 14. The electrode assembly 122 preferably comprises a cylindrical inner electrode 128 and a cylindrical outer electrode 126. The inner electrode 128 is preferably solid or any hollow in the inner electrode is sealed so that fluid does not enter any such hollow. The cylindrical shape of the inner electrode 128 and the outer electrode 126 is preferred and results in better performance than obtained with electrodes of other shapes, e.g., elongated flat panels.

The diameter A of the inner electrode 128 is preferably about one-half inch but the diameter A of the inner electrode can be selected by those skilled in the art in accordance with the particular application for the electrode using the information contained herein. The outer electrode 126 should be of a generally cylindrical shape and preferably be fabricated from titanium or niobium having a thickness (indicated at B in FIG. 15) which ensures that the inner electrode is shielded from potentially physical damage. As will be appreciated, titanium and niobium provide the advantage of resistance against corrosion which further prevents the introduction of harmful substances into the fluid being electrolyzed.

Still referring to FIG. 15, the space, indicated at C, between the inner electrode 128 and the outer electrode 126 does not exceed a maximum value. In contrast to previously available devices which separate the electrodes by greater distances and then utilize higher voltages to obtain the desired electrolyzation, the present invention keeps the electrode spacing small and obtains improved performance over other schemes. It is preferred that the space between the inner electrode 128 and the outer electrode 126 be not more than about one-half (½) inch; it is more preferred that the space between the inner electrode 128 and the outer electrode 126 be not more than about three-eights (⅜) inch; and, it is most preferred that the space between the inner electrode 128 and the outer electrode 126 be not more than about one-quarter (¼) inch.

Reference will next be made to FIG. 15A which is a side cross sectional view of the electrode assembly taken along line 3-3 in FIG. 15. As seen in FIG. 15A, the outer electrode 126 extends above the inner electrode 128 to provide improved electrical performance and physical protection. The outer electrode 126 is attached to the floor 124 by way of bolts 130, which extend through bores provided in the floor 124, and accompanying nuts. An electrical connection is made to the outer electrode 126 by a lead 136 attached to the bolt and nut. The lead 136 is attached to one of the terminals 110 or 112. Similarly, an electrical connection is made to the inner electrode 128 by a lead 134 which is held in place by a nut attached to a threaded stud extending from the bottom of the inner electrode and through a bore provided in the floor 124. The lead 134 is attached to the remaining one of the terminals 110 or 112. The leads 134 and 136 are kept insulated from any fluid which is present in the fluid vessel 116.

It is preferred that the inner electrode 128 function as the anode while the outer electrode function as the cathode when electrolyzing fluids and the power supply 102 and the terminals 110 and 112 should be properly arranged to carry this out.

It is recognized in the art that the anode is subject to destructive forces during electrolysis. In the prior art, the anode of an electrode assembly may dissolve to the point of being inoperative and may need to be replaced very often. Critically, as the anode of an electrode assembly dissolves, the metallic components of the anode are dispersed into the fluid. If the fluid is a saline solution which will be used to treat physiological fluids, toxic substances dispersed into the solution, such as the materials comprising the anode, may be harmful or dangerous to the person who expects to be benefited from the treatment.

Of all the possible materials for fabrication of the anode, the art recognizes that platinum is the least likely to be dissolved when used as an anode. Unfortunately, the cost of platinum precludes the use of an anode which consists entirely of platinum. Thus, it is common in the art to utilize another metal as a base for the anode with a layer of platinum being placed on surfaces which contact the fluid to be electrolyzed.

The present invention advantageously utilizes an inner electrode 128, i.e., an anode, which includes a base of titanium, and even more preferably niobium (also known as columbium), upon which a layer of platinum is provided wherever fluid contacts the anode. Significantly, niobium is a relatively good electrical conductor having a conductivity which is about three times greater than the conductivity of titanium. Moreover, if the base metal is exposed to the fluid, such as if a pinhole defect develops, toxic products are not produced by the contact between niobium and the fluid. Moreover, the high breakdown voltage in saline solution of the oxide which forms when a niobium base receives a layer of platinum provides further advantages of the present invention.

Upon a base of niobium, a layer of platinum is formed on the anode. The layer of platinum is preferably formed using a technique referred to in the art as brush electrodeposition which can be carried out by those skilled in the art using the information set forth herein. Other techniques can also be used to form the platinum layer, such as tank (immersion) electrodeposition, vapor deposition, and roll bonding, but brush electrodeposition is preferred because of its superior adhesion and resulting less porosity than other economically comparable techniques.

The thickness of the platinum layer is preferably greater than about 0.02 mils and is most preferably greater than about 0.06 mils, and up to about 0.20 mils. The combination of using niobium as a base for the anode of the electrode assembly and utilizing brush electrodeposition provides that the platinum layer can be much thinner than otherwise possible and still provide economical and reliable operation. It will be appreciated by those skilled in the art, that even with an anode fabricated in accordance with the present invention replacement of the anode, which preferably comprises the inner electrode 128 represented in FIG. 15A, may be necessary after a period of use. The construction of the embodiments of the present invention facilitate replacement of the inner electrode 128 and the outer electrode 126 when it becomes necessary.

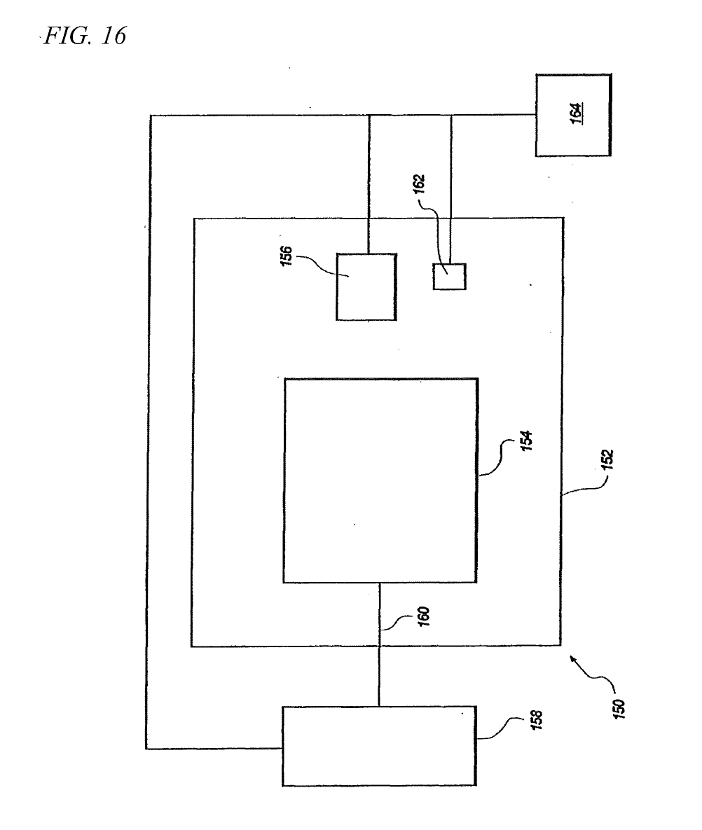

Represented in FIG. 16 is a block diagram of a second presently preferred embodiment, generally represented at 150, of the present invention. The embodiment represented in FIG. 16 is particularly adapted for treating large quantities of saline solution. Represented in FIG. 16 is a tank 152 in which the saline solution is electrolyzed. An electrode assembly 154 is provided in the tank and is preferably immersed into the solution. A power supply 158, capable of providing sufficient current at the proper voltage, is connected to the electrode assembly via a cable 160.

Also represented in FIG. 16 is a circulation device 156 which optionally functions to circulate the solution within the tank 152. A sensor 162 is also optionally provided to measure the progress of the electrolyzation of the solution in the tank 152, for example by measuring the pH of the solution. The sensor may preferably be an ion selective electrode which can be chosen from those available in the art. Other sensors, for example chlorine, ozone, and temperature sensors, may also be included within the scope of the present invention. A control unit 164 is optionally provided to coordinate the operation of the power supply 158, the circulation device 156, and the sensor 162 in order to obtain the most efficient operation of the apparatus 150.

It will be appreciated that devices such as power supply 158, circulation device 158, sensor 162, and control unit 164 can be readily obtained from sources in the industry and adapted for use with embodiments of the present invention by those skilled in the art using the information contained herein. In particular, the control unit 164 is preferably a digital microprocessor based device accompanied by appropriate interfaces all allowing for accurate control of the operation of the apparatus 150. It is also within the scope of the present invention to include structures to prevent contamination of the treated solution by contact with nonsterile surfaces and by airborne pathogens both during treatment and while the fluid is being transferred to the apparatus and being withdrawn from the apparatus.

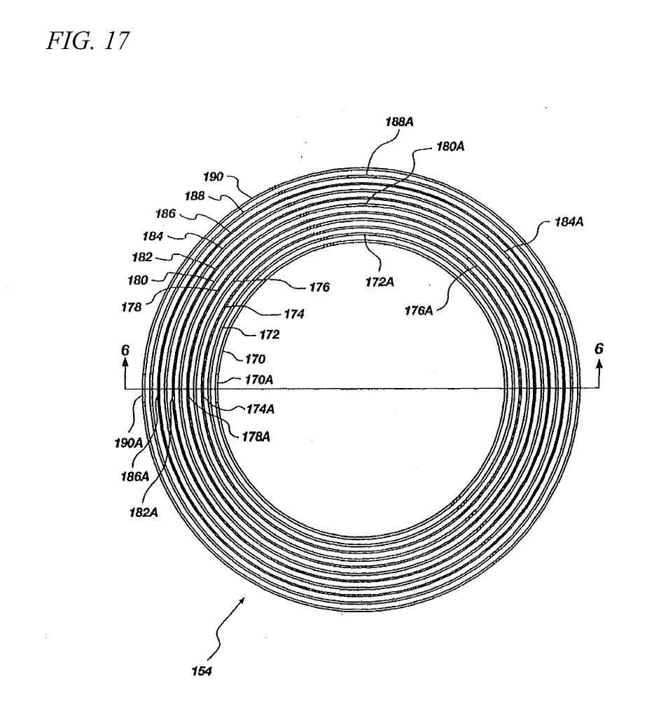

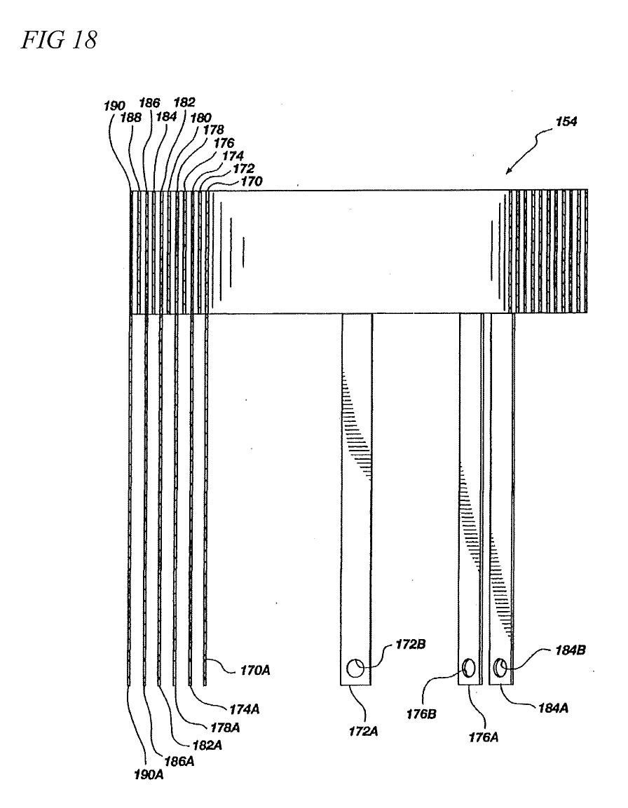

Reference will next be made to FIGS. 17 and 18 which are a top view and cross sectional view, respectively, of an electrode assembly, generally represented at 154, which is preferred for use in the apparatus represented in FIG. 16. As can be seen best in FIG. 17, the electrode assembly 154 includes a plurality of concentrically arranged anodes and cathodes. The cylindrical shape and concentric arrangement of the electrodes represented in FIG. 17 provides for the most efficient operation. The number of electrodes which are included can be selected according to the application of the apparatus. For example, the number of electrodes may be six, seven, eight, the eleven represented in FIGS. 17 and 18, or more.

In FIG. 17, electrodes 170, 174, 178, 182, 186, and 190 preferably function as cathodes and are preferably fabricated in accordance with the principles set forth above in connection with the outer electrode represented at 126 in FIGS. 14-15A. Furthermore, in FIG. 17 electrodes 172, 176, 180, 184, and 188 function as anodes and are preferably fabricated in accordance with the principles set forth above in connection with the inner electrode represented at 128 in FIGS. 14-15A.

In the cross sectional side view of FIG. 18 a plurality of tabs extend from the cylindrical electrodes 170, 172, 174, 176, 178, 180, 182, 184, 186, and 190 to facilitate making an electrical connection thereto. Provided below in the following Table are the relationship between the tabs illustrated in FIG. 18 and the electrodes.

Relationship between the tabs illustrated in FIG. 18

| Electrode | Tab | Function |
|---|---|---|
| 170 | 170A | Cathode |
| 172 | 172A | Anode |
| 174 | 174A | Cathode |
| 176 | 176A | Anode |
| 178 | 178A | Cathode |
| 180 | 180A | Anode |
| | (Not | |
| | illustrated | |
| | in FIG. 18) | |
| 182 | 182A | Cathode |
| 184 | 184A | Anode |
| 186 | 186A | Cathode |
| 188 | 188A | Anode |
| 190 | 190A | Cathode |
| | (Not | |
| | illustrated | |
| | in FIG. 18) | |

Using the tabs 170A, 172A, 174A, 176A, 178A, 180A, 182A, 184A, 186A, 188A, and 190A, those skilled in the art can provide the necessary electrical connections to the electrodes 170, 172, 174, 176, 178, 180, 182, 184, 186, and 190 and can also provide numerous structures to prevent contact between the tabs and the fluid to be treated. Each of the tabs illustrated in FIG. 18 are provided with an aperture, such as those represented at 172B, 176B, and 184B, which receive a wiring connector.

While the apparatus described in Example 3 herein has many uses, the most preferred use of the apparatus described herein is subjecting sterile saline solution to electrolysis. The electrolyzed saline solution can then be used to treat a patient. The saline solution preferably has an initial concentration in the range from about 0.25% to about 1.0% NaCl which is about one-fourth to full strength of normal or isotonic saline solution. According to Taber's Cyclopedic Medical Dictionary, E. A. Davis, Co. 1985 Ed., an “isotonic saline” is defined as a 0.16 M NaCl solution or one containing approximately 0.95% NaCl; a “physiological salt solution” is defined as a sterile solution containing 0.85% NaCl and is considered isotonic to body fluids and a “normal saline solution;” a 0.9% NaCl solution which is considered isotonic to the body. Therefore, the terms “isotonic,” “normal saline,” “balanced saline,” or “physiological fluid” are considered to be a saline solution containing in the range from about 0.85% to about 0.95% NaCl. Moreover, in accordance with the present invention, a saline solution may be subjected to electrolysis at concentrations in the range from about 0.15% to about 1.0%.

It is preferred that one of the above described saline solutions be diluted with sterile distilled water to the desired concentration, preferably in the range from about 0.15% to about 0.35% prior to treatment in accordance with the present invention. This dilute saline solution is subjected to electrolysis using the embodiments of the present invention at a voltage, current, and time to produce an appropriately electrolyzed solution as will be described shortly. It is presently preferred to carry out the electrolysis reaction at ambient temperatures. In a more preferred embodiment the saline solution used with the apparatus of Example 3 is 9.1 gNaCl/1 L of water. In another preferred embodiment the saline solution used with the apparatus of Example 3 is 2. gNaCl/1 L of water.

The voltage and current values provided herein are merely exemplary and the voltage and current values which are used, and the time the saline solution is subject to electrolysis, is determined by many variables, e.g., the surface area and efficiency of the particular electrode assembly and the volume and/or concentration of saline solution being electrolyzed. For electrode assemblies having a different surface area, greater volumes of saline solution, or higher concentrations of saline solutions the voltage, current, or time may be higher and/or longer than those exemplary values provided herein. In accordance with the present invention, it is the generation of the desired concentration of ozone and active chlorine species which is important. Electrolyzation of the saline solution also results in other products of the electrolysis reaction including members selected from the group consisting of hydrogen, sodium and hydroxide ions. It will be appreciated that the interaction of the electrolysis products results in a solution containing bioactive atoms, radicals or ions selected from the group consisting of chlorine, ozone, hydroxide, hypochlorous acid, hypochlorite, peroxide, oxygen and perhaps others along with corresponding amounts of molecular hydrogen and sodium and hydrogen ions.

According to Faraday's laws of electrolysis, the amount of chemical change produced by a current is proportional to the quantity of electrons passed through the material. Also, the amounts of different substances liberated by a given quantity of electrons are proportional to the chemical equivalent weights of those substances. Therefore, to generate an electrolyzed saline having the desired concentrations of ozone and active chlorine species from saline solutions having a saline concentration of less than about 1.0%, voltage, current, and time parameters appropriate to the electrodes and solution are required to produce an electrolyzed solution containing in the range from about 5 to about 100 mg/L of ozone and a free chlorine content in the range from about 5 to about 300 ppm. For in vitro use these solutions can be utilized without further modification or they can be adjusted as desired with saline or other solutions. Prior to in vivo use, the resulting solution may be adjusted or balanced to an isotonic saline concentration with sufficient hypertonic saline, e.g., 5% hypertonic saline solution.

In general, the electrolyzed solutions produced using the apparatus described herein, which are referred to as microbicidal solutions, will have an ozone content in the range from about 5 to about 100 mg/L and an active chlorine species content in the range from about 5 to about 300 ppm. More preferably the ozone content will be in the range from about 5 to about 30 mg/L and the active chlorine species content will be in the range from about 10 to about 100 ppm. Most preferably the ozone content will be in the range from about 9 to about 15 mg/L and the active species content will be in the range from about 10 to about 80 ppm. By active chlorine species is meant the total chlorine concentration attributable to chlorine content detectable by a chlorine ion selective electrode and will be selected from the group consisting of chlorine, hypochlorous acid and hypochlorite ions or moieties.

The pH of the solution is preferably in the range from about 7.2 to about 7.6 and, when used for intravenous administration, most preferably in the range from about 7.35 to about 7.45 which is the pH range of human blood. An effective amount of the resulting balanced microbicidal saline solution is administered by appropriate modes, e.g., intravenously, orally, vaginally or rectally and may vary greatly according to the mode of administration, condition being treated, the size of the warm-blooded animal, etc.

Particular dosages and methods of administration, as well as additional components to be administered, can be determined by those skilled in the art using the information set forth herein and set forth in the U.S. patent documents previously incorporated herein by reference. As explained in the cited U.S. patent documents, although it is known that electrolyzed saline solutions possess in vitro microbicidal activity it has long been thought that components in the electrolyzed solution, such as ozone and chlorine, are toxic to warm blooded animals and should not be utilized for in vivo purposes. It has now been found, however, that saline solutions, which have been subjected to electrolysis to produce finite amounts of ozone and active chlorine products, can be injected into the vascular system to create a reaction to assist in the removal, passivation, or destruction of a toxin.

In order to arrive at the preferred end product, electrolyzed saline solution using the apparatus illustrated in FIGS. 14-15A, about a 0.33% (about one third physiologically normal) saline solution is placed in the fluid vessel 116 (FIG. 14) and the apparatus is operated for about 5 to 15 minutes with a voltage between the electrodes being maintained in the range from about 10 volts to about 20 volts with a current flow maintained in the range from about 5 to about 20 amps.

In one example, the cell described in Example 3 operated for 1 hour at 40 C using 3 Amps with a saline solution of less than 0.35% saline.

In one example, the cell described in Example 3 operated for 1 hour at 40 C using 3 Amps with a saline solution of less than 1.0% saline.

In one example, the cell described in Example 3 operated for 3 minutes at 23 C using 3 Amps with a saline solution of less than 0.35% saline.

In one example, the cell described in Example 3 operated for 3 minutes at 23 C using 3 Amps with a saline solution of less than 1.0% saline.

As one example of the use of the embodiment of FIGS. 14-15A, a 0.225% saline solution is subjected to a current of 3 amperes at 20 volts (DC) for a period of three minutes. A 17 ml portion of this electrolyzed solution is aseptically diluted with 3 mls of a sterile 5% saline resulting in a finished isotonic electrolyzed saline having an active ozone content of 12.+−0.2 mg/L and an active chlorine species content of 60.+−0.4 ppm at a pH of 7.4.

It will be appreciated that the low voltages used in accordance with the present invention are preferably not greater than forty (40) volts DC or an equivalent value if other than direct current is used. More preferably, the voltages used in accordance with the present invention is not more than about thirty (30) volts DC. The use of low voltages avoids the problem of production of undesirable products in the fluid which can result when higher voltages are used. In accordance with the present invention, the close spacing of the electrodes facilitates the use of low voltages.

In another example, to show that the embodiment of FIGS. 14-15 can be used to effectively carry out electrolysis in saline solutions up to about 1% in concentration, the electrolysis reaction is carried out at saline concentrations of 0.3, 0.6 and 0.9%, respectively. The active chlorine species Cl₂ and ozone O₃ contents were measured and are provided in the table below:

Cl₂ and O₃ Content from Salines at Varying Concentrations

| Saline | Cl₂ | |
|---|---|---|
| Concentration | Concentration | O₃ Concentration |
| (% NaCl) | (ppm) | (mg/mL) |
| 0.3 | 129 | 21.8 |
| 0.6 | 161 | 26.6 |
| 0.9 | 168 | 28.0 |

As can be seen from the above table, the resulting electrolyzed saline solution includes active components which are within the parameters required for effective treatment.

It will be appreciated that the features of the present invention, including the close electrode spacing, the low voltages used, and the materials used to fabricate the electrodes, result in an apparatus which provides unexpectedly better results than the previously available devices and schemes.

### Example 4

A saline solution was made with the apparatus of Example 3 wherein the solution was electrolyzed for 3 min at 3 amps and such that the solution being electrolyzed had 9.1 g NaCl/L of purified water. The product made accordingly is called RXN-1. The RXN-1 product was tested for superoxides and hypochlorites as described herein. Specifically, the presence of superoxides was tested with the Nanodrop 3300 and R-phycoerytherin (R-PE) as the reagent and the presence of hypochlorites was tested with the Nanodrop 3300 and aminophenyl fluorescein (APF) as the reagent. The tests revealed the presence of both superoxides as well as hypochlorites. The superoxides were tested as an amount relative to the amount of superoxides that are present in a sample made according to Example 1. That is, superoxides were tested as an amount relative to the amount of superoxides when a total of 1,000 gallons of salinated water was electrolyzed with a total of 56 amps running through the electrodes and further wherein the electrolyzing occurred at 4.5-5.8° C. The amount of superoxides present in the RXN-1 product was 130% of the amount of superoxides present in a sample made according to Example 1. Similarly, the hypochlorites were tested as an amount relative to the amount of hypochlorites that are present in a sample made according to Example 1. That is, hypochlorites were tested as an amount relative to the amount of hypochlorites when a total of 1,000 gallons of salinated water was electrolyzed with a total of 56 amps running through the electrodes and further wherein the electrolyzing occurred at 4.5-5.8° C. The amount of hypochlorites present in the RXN-1 product was 82% of the amount of hypochlorites present in a sample made according to Example 1.

### Example 5

A saline solution was made with the apparatus of Example 3 wherein the solution was electrolyzed for 3 min at 3 amps and such that the solution being electrolyzed had 2.8 g NaCl/L of purified water. The product made accordingly is called RXN-2. The RXN-2 product was tested for superoxides and hypochlorites as described herein. Specifically, the presence of superoxides was tested with the Nanodrop 3300 and R-phycoerytherin (R-PE) as the reagent and the presence of hypochlorites was tested with the Nanodrop 3300 and aminophenyl fluorescein (APF) as the reagent. The tests revealed the presence of both superoxides as well as hypochlorites. The superoxides were tested as an amount relative to the amount of superoxides that are present in a sample made according to Example 1. That is, superoxides were tested as an amount relative to the amount of superoxides when a total of 1,000 gallons of salinated water was electrolyzed with a total of 56 amps running through the electrodes and further wherein the electrolyzing occurred at 4.5-5.8° C. The amount of superoxides present in the RXN-2 product was 120% of the amount of superoxides present in a sample made according to Example 1. Similarly, the hypochlorites were tested as an amount relative to the amount of hypochlorites that are present in a sample made according to Example 1. That is, hypochlorites were tested as an amount relative to the amount of hypochlorites when a total of 1,000 gallons of salinated water was electrolyzed with a total of 56 amps running through the electrodes and further wherein the electrolyzing occurred at 4.5-5.8° C. The amount of hypochlorites present in the RXN-2 product was 80% of the amount of hypochlorites present in a sample made according to Example 1.

Power Sources

Readily available electricity, such as that which comes from a wall socket, is brought to a terminal strip. This terminal strip, also known as a terminal block, acts like a surge protector allowing a number of electrical connections from the strip to other devices. For example, the terminal strip can be an interface for electrical circuits. The terminal strip can be connected to a ground and/or a current transformer. A transformer can be used to measure electric currents. The terminal strip can also be connected to a potentiometer. The potentiometer measures voltage across an electrical system and can be used to aid in adjusting the voltage. For example a dial can be connected to the potentiometer so that the operator may adjust the voltage as desired.

Another transformer can be connected to the potentiometer, which can then be operably connected to a rectifier. Rectifiers in general convert alternating current (AC) to direct current (DC). One specific type of rectifier which suits the invention well is a bridge rectifier. Converting the waveform into one with a constant polarity increases the voltage output. This waveform is called a full wave rectified signal. Once the waveform and voltage are configured as desired, the DC shunt can provide a means for bringing electricity to different devices such as the electrodes, monitors and other operational instruments.

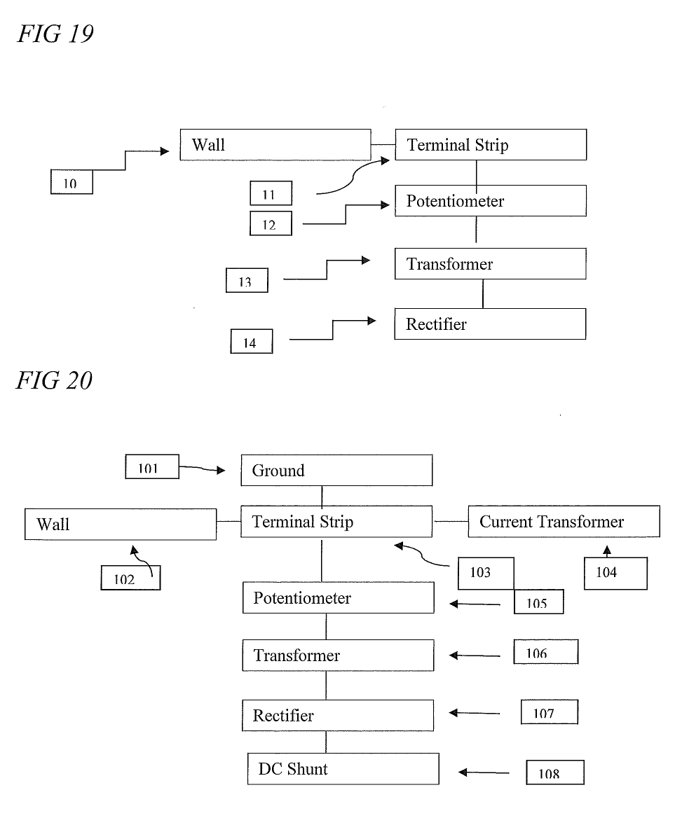

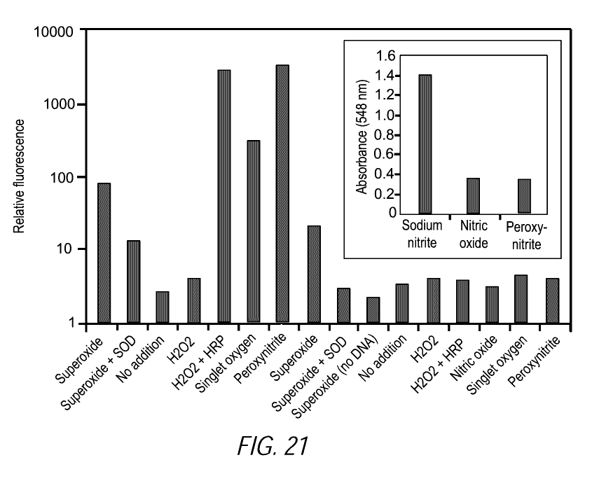

FIG. 19 diagrams an example of a power source which can be used in the invention. Electricity comes in from the wall 10 and is met by a terminal strip 11. Terminal strip 11 is in operable communication with a potentiometer 12, and a current transformer 13. Potentiometer 12 is in operable communication with the transformer 13. The transformer 13 is in operable communication with a rectifier 14.

FIG. 20 diagrams an example of a power source which can be used in the invention. Electricity comes in from the wall 102 and is met by a terminal strip 103. Terminal strip 103 is in operable communication with a potentiometer 105, a grounding means 101 and a current transformer 104. Potentiometer 105 is in operable communication with the transformer 106. The transformer 106 is in operable communication with a rectifier 107. Rectifier 107 is in operable communication with a DC shunt 108.

Determination of ROS Levels Against a Known Standard

The measurement of concentrations of ROS, particularly a superoxide, inside the solutions has been done by means of a fluoro spectrometer, Nanodrop 3300, and three varieties of fluorescent dyes, R-Phycoerytherin (R-PE), Hydroxyphenyl fluorescein (HPF) and Aminophenyl fluorescein (APF), that are commonly used to determine relative ROS concentrations inside active biological systems and cells. The molecules in these dyes change shape, and therefore fluoresce only when exposed to molecular components in ROS. The resulting change in fluorescence can then be detected by the fluoro spectrometer and can be related to the concentration of ROS present. ROS concentrations in electrolyzed saline solutions (ESS) solutions are verified and detected by either APF or R-PE fluorescent dyes, both of which produce entirely consistent measurements of relative concentrations of ROS in various concentrations and dilutions of ESS solutions. ROS measurements in ESS solutions have been linked using R-PE fluorescent dye, to the reaction of this dye to regulated concentrations of 2/2′-Axobis(2-methylpropionamide)dihidrochloride, a molecule that produces known amounts of ROS. This is not an absolute measurement, but it relates ROS in ESS to amounts of a known producer of ROS.

These fluorescent dyes are often used in combination with a fluorescence microscope to create high-resolution images of the build-up of ROS (oxidative stress) inside individual living cells. These dyes have been shown to specifically be sensitive to concentrations of ROS regardless of complex surrounding chemical environments.

Although APF and R-PE dyes are capable of measuring relative ROS concentrations in ESS solutions, no known absolute standard concentration for stabilized ROS in pure saline solutions exists. Furthermore, discrepancies in the decay time of these fluorescent dyes make measuring standardized amounts of ROS in other solutions incompatible with measuring those found in ESS. This may be due, in part, to the molecular complexes in ESS solutions that keep the ROS concentration stable, effectively shielding the free radicals from readily reacting with the dyes. The standard for ROS concentration in ESS solutions is therefore measured relative to the ROS concentration in a standardized solution that has been used in all of the antimicrobial and toxicity studies to date, both published and unpublished. Methods to measure absolute ROS concentrations in ESS solutions are actively being pursued.

The regulated amounts of ROS, thus measured, inside a variety of the ESS solutions produced by various embodiments of this invention have been shown to be stable, consistent and predictable, sufficient for therapeutic applications.

The development of a phycobiliprotein fluorescence quenching assay for the routine determination of ROS content in ASEA has been successful and is used routinely to monitor production quality for ROS levels. The assay has the following characteristics: ease of use, sensitivity, and quantitation. The assay is linear over a 2 log 10 range of ROS concentrations. For a compositions comprising RXNs, the starting saline was used as a negative control, AAPH (2,2′-Azobis(2-amidinopropane) dihydrochloride which is a standard ROS generating compound) served as a positive control and allowed the generation of a standard curve, and the compositions comprising RXNs or other samples comprised the unknowns.

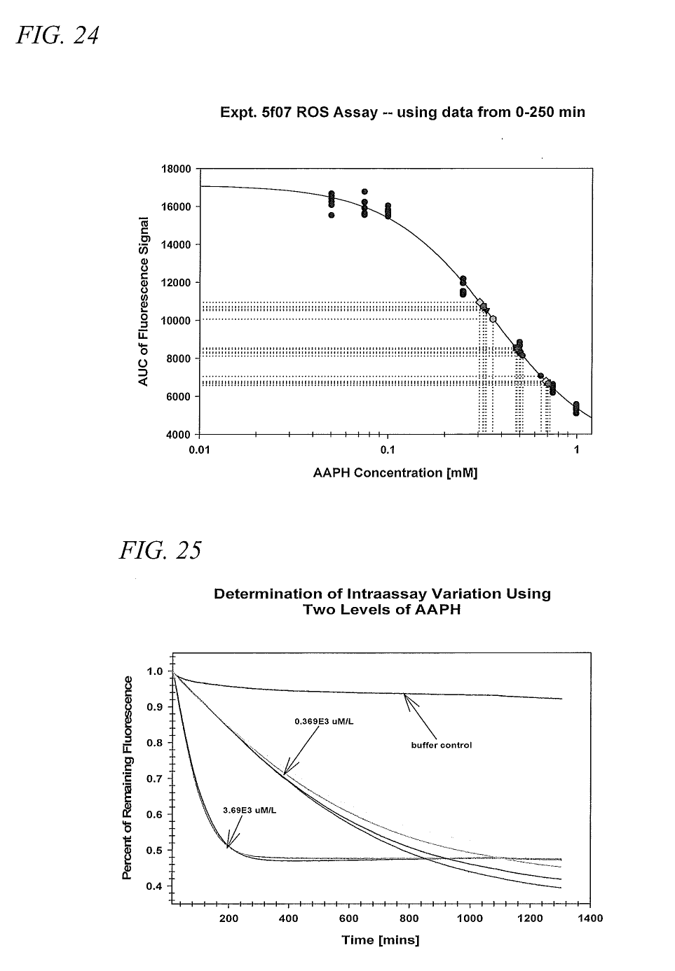

For the purposes of this work, we determined the oxygen radical content of our health benefiting product. In the assay described below, R-Phycoerythrin [an algal protein] is exposed to varying levels of a standard ROS generating compound [AAPH] wherein the level of fluorescence quenching is logarithmically related to ROS content. This provides a standard curve from which to estimate the ROS content of unknown samples. The levels of ROS in the unknown samples are expressed as mM equivalents of AAPH. FIG. 24 shows the concentration of AAPH.

Materials and Methods:

PHYCOERYTHRIN and R-PHYCOERYTHRIN: were purchased from Sigma Chemical Corporation, St. Louis, Mo.

AAPH: 2,2′-azobis(2-amidino-propane) dihydrochloride was purchased from Wako Chemicals USA, Richmond, Va. This compound generates ROS upon contact with water.

FLUORESCENCE READER: an 8 or 16 place fluorescence reader manufactured by Pacific Technologies, Redmond, Wash. was used to detect the fluorescence signal from the phycoerythrins. Temperature was controlled at 37 C during a 12-20 hr. experimental run. The samples were interrogated every 0.5 to 2 min where each sample interrogation was comprised of 1024 lamp flashes from a LED whose emission spectra was appropriate from the excitation spectra of R-Phycoerythrin. Proper cut-off filters were employed to detect the fluorescence emissions of the phycoerythrins.

DATA ANALYSES: All data is captured in real time. The data contained in the worksheet can be manipulated to determine the relative change of fluorescence over the time course of the experiment and subsequently, SigmaPlot Pro v. 7 software [SPSS Software, Chicago, Ill.] is used to determine the area under the curve. Area under the curve [AUC] analysis is appropriate since Cao, Cao et al. Comparison of different analytical methods for assessing total antioxidant capacity of human serum. Clinical Chemistry June 1998 vol. 44 no. 6 1309-1315 which is hereby incorporated by reference in its entirety, and colleagues have demonstrated that in this method both the inhibition time and degree of inhibition of fluorescence by free radicals are considered. The area under the curve [AUC] are plotted against the log 10 mM AAPH concentration to provide a standard curve from which to estimate the levels of ROS in unknown samples.

Detailed Methods:

Step a. 300 uL of phosphate buffer, pH 7.0, 100 mM is added to ½″ glass vials.

Step b. 15 ug of R-Phycoerythrin in 15 uL of phosphate buffer is added to the materials in Step a. The vials are capped and placed into the wells of the fluorescence reader for 15 min prior to the addition of a saline control, ASEA or AAPH solutions. During this period, fluorescence values are collected from which to calculate a 100% value. This value is then used in subsequent calculations to determine a relative fluorescence signal value for the standard curves.

1 mg of AAPH is added to 1 ml of phosphate buffer and 10-fold dilutions are made to provide at least a 3 log 10 range of AAPH concentrations. Similarly, ASEA solutions are diluted and added to appropriate vials in Step b.

100 uL of the materials in Step a are added to the appropriate vials in Step b. The vials are mixed and replaced into the reader for up to an additional 12 to 20 hrs of evaluation.

RESULTS: As shown in FIG. 24, as the concentration of AAPH decreased from 1.00 mM to 0.050 mM, there was as concomitant increase in the normalized AUC. Buffer control [not shown] revealed that over time there is a spontaneous loss of fluorescence signal, although this loss represents only ˜8% of the original signal.

The data represented in FIG. 25 shows intra-assay variability of two concentrations of AAPH. Using SigmaStat v 2.01 software, the following mean, Std Deviation and Relative Std Deviation were calculated and are presented in Table 1. The data shows that the variation for each concentration the variation among replicates ranged from ˜0.1% to 4% variation [Rel. Std. Dev.]. These data suggest that fluorescence quenching assay is capable of producing small variations among triplicate or quadruplicate samples over a 10-fold range of AAPH concentrations.

TABLE 1
Intraassay Variability

| | AUC Values | | | | |
|---|---|---|---|---|---|
| | | Mean | | | % Rel. |
| AAPH Concentration | N | AUC | Std. Dev. | Std. Error | Std. Dev. |
| 3.69 mM | 3 | 653 | 1.07 | 0.62 | 0.15 |
| 0.369 mM | 4 | 804 | 31.7 | 15.0 | 3.7 |

Table 2 shows the results of the analyses of ASEA solutions prepared by MDI and filtered through 0.2 u Supor membrane to ensure sterility prior to clinical application. It is clear that the ASEA from different production lots are similar in their ROS content. Statistical analysis supported this observation [p=0.272]. The most important point is the observation that filtration through a 0.2 u Supor membrane does not decrease the ROS content of ASEA.

Table 2. ROS Content of ASEA Filtered and Unfiltered Through 0.2 Supor Membrane

TABLE 2

| Treatment | N | Mean AUC | Std. Dev. | Std. Error | % Rel. Std. Dev. |
|---|---|---|---|---|---|
| Unfiltered | 4 | 589.7 | 65.8 | 32.9 | 5.5 |
| Filtered | 4 | 646.3 | 66.3 | 33.1 | 5.1 |

The levels of variance [Rel. Std. Dev.] reported by us is similar to that reported by Cao and colleagues.

In Table 3, data from a typical analysis is illustrated. Saline [negative control] always contained less than 0.1 mM AAPH equivalents of ROS whereas ASEA always contained >1.0 mM ROS.

TABLE 3
ROS Content of ASEA and Saline

| | | ROS Content |
|---|---|---|
| ASEA or Saline | | mM AAPH |
| Samples | Mean AUC | equivalents |
| ASEA | 479 | 3.3 |
| ASEA | 543 | 2.2 |
| ASEA | 441 | 4.5 |
| ASEA | 523 | 2.98 |
| ASEA | 516 | 3.2 |
| Saline | 974 | 0.095 |
| Saline | 956 | 0.075 |

The above shows a known concentration of a standard, AAPH, as 653 and 804 when tested at 3.69 mM and 0.369 mM respectively. A compositions comprising RXNs showed a AUC of between 441-543.

The measurement of concentrations of ROS inside the solutions can be done by means of a fluorospectrometer, Nanodrop 3300, and three varieties of fluorescent dyes, R-Phycoerytherin (R-PE), Hydroxyphenyl fluorescein (HPF) and Aminophenyl fluorescein (APF), all of which are commonly used to determine relative ROS concentrations inside active biological systems and cells. The molecules in these dyes change shape, and therefore fluoresce only when exposed to molecular components in ROS. The resulting change in fluorescence can then be detected by the fluorospectrometer and can be related to the concentration of ROS present. ROS concentrations in a compositions comprising RXNs can be verified and detected by either APF or R-PE fluorescent dyes, both of which produce entirely consistent measurements of relative concentrations of ROS in various concentrations and dilutions of RXNs. The ROS measurements in a compositions comprising RXNs have been linked, using R-PE fluorescent dye, to the reaction of this dye to regulated concentrations of 2/2′-Axobis(2-methylpropionamide) dihidrochloride, a molecule that produces known amounts of ROS.

Superoxide Testing

Superoxides were tested with the NanoDrop 3300 and R-PE as the reagent for the three samples.

The intensity of the fluorescence indicates the amount of ROS in the sample. This dye, R-PE, is toxic, expensive, must be kept refrigerated, degrades in strong blue light, such as a fluorescent bulb, and is time sensitive. The following steps were taken:

The ND-3300 software was called up, the “Other Fluorophores” button was clicked and the “R-PE 50 uM Activated” option was selected.

The ND-3300 was blanked: 2 uL (1 drop) of deionized water was placed using a pipette on the measurement pedestal and the arm was carefully closed. The “Blank” button was clicked and the ND-3300 took a “blank” measurement, thereby calibrating the ND-3300.

The samples were prepared by pipetting 10 ml deionized water into each one of the large (15 ml) test tubes required for the test. One test tube will be required for each sample to be tested.

The test tubes were labeled by cutting out squares of sticky-back label stock, large enough to fit over the mouth of the test tubes, and by writing the number “1”, “2” and “3” on the label. The labels were placed covering the mouth of the test tubes to both identify them and to keep the liquids from evaporating.

10 ul of the R-PE fluorescent dye was apportioned into each of the test tubes by following these steps: turning off the lights, taking the previously prepared R-PE dye test tube out of the refrigerator [this test tube was previously prepared by putting 2 ul of the concentrate from the commercial R-PE vial inside 5 ml deionized water (a phosphate buffer is not needed)]. The prepared test tube was placed in the rack with the others. This dye is toxic and is sensitive to light so these steps should be done quickly, with lab coat, gloves and goggles. With a clean pipette, 10 ul of the prepared R-PE dye was add into each of the test tubes. The R-PE was placed back in the test tube back in the refrigerator.

The test tubes were mixed well using a mixing pipette which was place into each of the test tubes, 2-3 ml were drawn out and then quickly pushed back in, allowing some bubbles to escape to better agitate the contents of the test tubes. This was repeated three to four times for each tube. At this point, it is necessary to have separate mixing pipette heads for each tube. The test tubes were allowed to sit for least 30 min. after mixing.

The initial pre-sample measurements were taken on all of the test tubes: The ND-3300 was blanked using the procedures outlined above. A folded Kimwipe was used to blot the last sample droplet off the lower and upper pedestals before loading a new drop to be analyzed. A descriptive name for the sample was typed into the Sample ID field in the software. 2 ul of test tube #1 was loaded onto the pedestal, the arm was carefully closed and the “measure” button pressed. Three measurements were taken of the sample in test tube #1. This procedure was repeated for the next two samples. Specifically, the Sample ID field was changed to reflect the descriptive name of the sample in the second test tube. And then three (3) measurements were taken from the second test tube also. This step was done until all test tubes were analyzed. When R-PE was activated, the RFU readings shown were between the 100 and 2000.

A compositions comprising RXNs was added to the test tubes: This procedure was carefully timed. The R-PE dye is only accurate for less than 30 minutes after activation and therefore all measurements must be acquired after the same amount of exposure time. 10 ul of a compositions comprising RXNs sample #1 was added to test tube #1 and immediately thereafter a timer was set for three (3) minutes. Then the test tube #1 was mixed with a pipette. This step was repeated for all three samples.

At 6 hrs post addition of the first a compositions comprising RXNs sample to a test tube, measurements were taken from every test tube in the following manner. The ND-3300 was blanked, the pedestals were blotted and the “Sample ID” for test tube #1 was typed in. After three (3) minutes, using a sampling pipette, a 2 ul drop was taken from test tube #1 and place it on the pedestal and the measure button was pressed. This process was repeated until all of the test tubes were measured.

The data was cleaned up by pressing the “Show Report” button so that all of the data that has been taken so far was displayed. The data was then saved and analyzed.

Hypochlorite Testing

Hypochlorites were tested with the NanoDrop 3300 Fluorospectrometer and APF as the reagent.

The ND-3300 software was called up, the “Other Fluorophores” button was clicked and the “APF 50 uM Activated” option was selected.

The ND-3300 was blanked: 2 uL (1 drop) of deionized water was placed using a pipette on the measurement pedestal and the arm was carefully closed. The “Blank” button was clicked and the ND-3300 took a “blank” measurement, thereby calibrating the ND-3300.

The samples were prepared by pipetting 10 ml deionized water into each one of the large (15 ml) test tubes required for the test. One test tube will be required for each sample to be tested.

The test tubes were labeled by cutting out squares of sticky-back label stock, large enough to fit over the mouth of the test tubes, and by writing the number “1”, “2” and “3” on the label. The labels were placed covering the mouth of the test tubes to both identify them and to keep the liquids from evaporating.

10 ul of the APF fluorescent dye was apportioned into each of the test tubes by following these steps: turning off the lights, taking the previously prepared APF dye test tube out of the refrigerator [this test tube was previously prepared by putting 2 ul of the concentrate from the commercial APF vial inside 5 ml deionized water (a phosphate buffer is not needed)]. The prepared test tube was placed in the rack with the others. This dye is toxic and is sensitive to light so these steps should be done quickly, with lab coat, gloves and goggles. With a clean pipette, 10 ul of the prepared APF dye was add into each of the test tubes. The APF was placed back in the test tube back in the refrigerator.

The test tubes were mixed well using a mixing pipette which was place into each of the test tubes, 2-3 ml were drawn out and then quickly pushed back in, allowing some bubbles to escape to better agitate the contents of the test tubes. This was repeated three to four times for each tube. At this point, it is necessary to have separate mixing pipette heads for each tube. The test tubes were allowed to sit for least 30 min. after mixing.

The initial pre-sample measurements were taken on all of the test tubes: The ND-3300 was blanked using the procedures outlined above. A folded Kimwipe was used to blot the last sample droplet off the lower and upper pedestals before loading a new drop to be analyzed. A descriptive name for the sample was typed into the Sample ID field in the software. 2 ul of test tube #1 was loaded onto the pedestal, the arm was carefully closed and the “measure” button pressed. Three measurements were taken of the sample in test tube #1. This procedure was repeated for the next two samples. Specifically, the Sample ID field was changed to reflect the descriptive name of the sample in the second test tube. And then three (3) measurements were taken from the second test tube also. This step was done until all test tubes were analyzed. When APF was activated, the RFU readings shown were between the 100 and 2000.

A compositions comprising RXNs was added to the test tubes: This procedure was carefully timed. The APF dye is only accurate for less than 30 minutes after activation and therefore all measurements must be acquired after the same amount of exposure time. 10 ul of a compositions comprising RXNs sample #1 was added to test tube #1 and immediately thereafter a timer was set for three (3) minutes. Then the test tube #1 was mixed with a pipette. This step was repeated for all three samples.

At 30 min. post addition of the first a compositions comprising RXNs sample to a test tube, measurements were taken from every test tube in the following manner. The ND-3300 was blanked, the pedestals were blotted and the “Sample ID” for test tube #1 was typed in. After three (3) minutes, using a sampling pipette, a 2 ul drop was taken from test tube #1 and place it on the pedestal and the measure button was pressed. This process was repeated until all of the test tubes were measured.

Packaging

The packaging process includes any type of packaging that does not contribute to the decay of the superoxides, hydroxyl radicals and OOH* (for example, containers should not contain metal oxides or ions). Pouches and bottles are preferred for ease of portability and acceptability in the market. However, any suitable packaging is applicable. Containers/packaging can be made of for example glass, polyethylene, polypropylene and the like. Specific examples include Bapolene HD2035, which is a high density polyethylene copolymer and Jade brand CZ-302 polyester. Table 4 shows the relative percentage of superoxides remaining after a 12 month period when the composition is packaged in a polyethylene bottle.

### Example 6

The rate of decay for superoxides, from a sample made according to Example 1, was tested over a 12 month period. That is, superoxides present in a sample made when a total of 1,000 gallons of salinated water is electrolyzed with a total of 56 amps running through the electrodes and further wherein the electrolyzing occurred at 4.5-5.8° C., according to Example 1, were tested for their relative amounts over a period of 12 months relative to a standard RFU control.

TABLE 4
1 Year Studies - shows a 3%/month decay rate over a 12 month period

| | | | | | | % Potency |
|---|---|---|---|---|---|---|
| | | RFU Average | RFU minus | Standard | | as compared to |
| Sample ID | RFU | per sample | control | deviation | % error | reference sample |
| RFU Control | 1743.7 | 1759.033 | | | | |
| Control | 1814.6 | | | | | |
| Control | 1718.8 | | | | | |
| Sample 1 | 985.6 | 986.1667 | 872.8667 | 6.169549 | 0.706815 | 1 |
| Sample 1 | 980.3 | | | | | |
| Sample 1 | 992.6 | | | | | |
| Sample 2 | 1044.8 | 1003.6 | 855.4333 | 35.68151 | 4.171162 | Baseline |
| Sample 2 | 982.7 | | | | | |
| Sample 2 | 983.3 | | | | | |
| Sample 3 | 981.7 | 988.3 | 870.7333 | 16.23915 | 1.864997 | 1.007618 |
| Sample 3 | 1006.8 | | | | | |
| Sample 3 | 976.4 | | | | | |
| Sample 4 | 1132.9 | 1121.133 | 737.9 | 12.56437 | 1.70272 | 0.853903 |
| Sample 4 | 1107.9 | | | | | |
| Sample 4 | 1122.6 | | | | | |
| Sample 5 | 1189.9 | 1182.2 | 676.8333 | 19.99475 | 2.954161 | 0.783236 |
| Sample 5 | 1197.2 | | | | | |
| Sample 5 | 1159.5 | | | | | |
| Sample 6 | 1269.3 | 1256.267 | 602.7667 | 26.47647 | 4.39249 | 0.697526 |
| Sample 6 | 1225.8 | | | | | |
| Sample 6 | 1273.7 | | | | | |

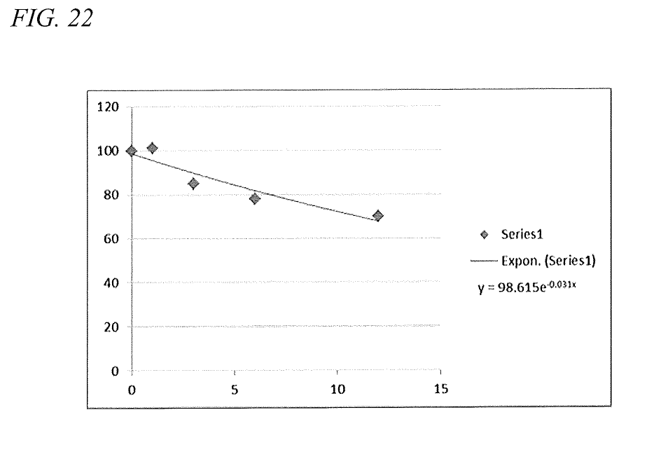

Table 4 provides data for the RFU control, Sample 1 which is a reference sample and Samples 2-6 which were taken at 1 month, 3 months, 6 months and 12 months. Table 4A shows the results as a percentage of remaining superoxides at 0, 1, 3, 6 and 12 months. This Table 4 is graphically represented in FIG. 22.

TABLE 4A

| Month X-axis | % Potency Y-axis |
|---|---|
| 0 | 100 |
| 1 | 101 |
| 3 | 85 |
| 6 | 78 |
| 12 | 70 |

### Example 7

Table 5 shows the relative percentage of superoxides remaining after a 13 month period when the composition is packaged in a polyethylene bottle and polyethylene pouch. In this Example, the composition tested was made according to the process of Example 6.

TABLE 5
13 Month Pouch v. Bottle

| | | | | | | % Potency |
|---|---|---|---|---|---|---|
| | | RFU Average | Standard | | RFU minus | as compared to |
| Sample ID | RFU | per sample | deviation | % error | control | reference sample |
| Control | 1687.9 | | | | | |
| 555 | 946.4 | 940.7667 | 9.157693 | 0.973429 | 1325.273 | 1 |
| 555 | 930.2 | | | | 1370.007 | |
| 555 | 945.7 | | | | | |
| 555-1 | 817.5 | 851.3 | 29.27781 | 3.439188 | 1414.74 | 1.067508 |
| 555-1 | 867.6 | | | | | |
| 555-1 | 868.8 | | | | | |
| 525b | 967.2 | 966.0333 | 10.3992 | 1.076484 | 1300.007 | 0.948905 |
| 525b | 955.1 | | | | | |
| 525b | 975.8 | | | | | |
| 524p | 983.1 | 975.7333 | 17.08576 | 1.751069 | 1290.307 | 0.941825 |
| 524p | 956.2 | | | | | |
| 524p | 987.9 | | | | | |
| 480 | 985.9 | 1006.333 | 19.12337 | 1.900302 | 1259.707 | 0.919489 |
| 480 | 1009.3 | | | | | |
| 480 | 1023.8 | | | | | |
| 479p | 1115.2 | 1153.5 | 45.22975 | 3.921088 | 1112.54 | 0.812069 |
| 479p | 1141.9 | | | | | |
| 479p | 1203.4 | | | | | |
| 408p | 1454.2 | 1501.633 | 62.98812 | 4.194641 | 764.4067 | 0.557958 |
| 408p | 1573.1 | | | | | |
| 408p | 1477.6 | | | | | |
| 347p | 1309.4 | 1327.833 | 39.24364 | 2.955464 | 938.2067 | 0.684819 |
| 347p | 1301.2 | | | | | |
| 347p | 1372.9 | | | | | |
| 347p | 1338.1 | | | | | |
| 314 | 1354.4 | 1348.567 | 16.82627 | 1.247715 | 917.4733 | 0.669685 |
| 314 | 1361.7 | | | | | |
| 314 | 1329.6 | | | | | |
| 313p | 1459.3 | 1444.033 | 13.25908 | 0.918198 | 822.0067 | 0.600002 |
| 313p | 1435.4 | | | | | |
| 313p | 1437.4 | | | | | |

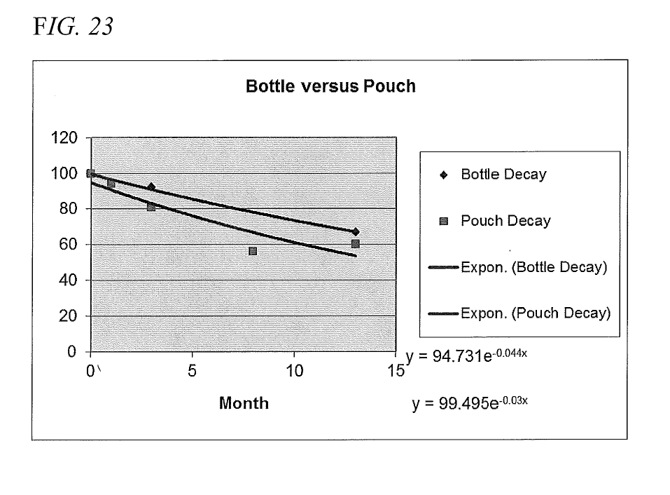

The above graph shows a 4.4% decay rate of the superoxide radical for the pouch and a 3% decay rate for the bottle over a 13 month period. Sample 555 is a reference sample, Sample 555-1 is a baseline sample, Sample 525b is a sample taken from a bottle after 1 month, Sample 524p is a sample taken from a pouch after 1 month, Sample 480 is a Sample taken from a bottle after 3 months, Sample 479p is a sample taken from a pouch after 3 months, Sample 408p is a sample taken from a pouch after 8 months, Sample 374p is a sample taken from a pouch after 11 months, Sample 314 is a sample taken from a bottle after 13 months and Sample 313p is a sample taken from a pouch after 13 months. Table 5A is a chart showing the percentage of remaining superoxides at 0, 1, 3, 8, 11 and 13 months in a bottle and a pouch type container. This Table 5 is graphically represented in FIG. 23.

TABLE 5A

| Month X-asis | % Potency Y-axis | % Potency Y-axis |
|---|---|---|
| 0 | 100 | 100 |
| 1 | 95 | 94 |
| 3 | 92 | 81 |
| 8 | | 56 |
| 11 | | 68 |
| 13 | 67 | 60 |

### Example 8

Borosilicate glass, such as those sold under the trade names of Kimax, Pyrex, Endural, Schott, or Refmex for example, are useful for packaging of a compositions comprising RXNs.

The presence of superoxides in a compositions comprising RXNs samples were tested after being stored in borosilicate glass bottles. The samples were made according to the process described in Example 6. Sample 397 had been stored for 24 months and Sample 512 had been stored for 20 months. Reference batch 1256 was made the same day as the test was run on all three samples. The Results are shown in Table 6.

TABLE 6
Glass Bottle ASEA Stability

| | | | Control − | % Potency as |
|---|---|---|---|---|
| | | average | average + | compared to |
| Sample | RFU | RFU | control loss | reference sample |
| 397 | 780.5 | 806.8 | 1193.2 | 93.1169 |
| | 819.5 | | | |
| | 820.4 | | | |
| 512 | 676.7 | 682.4666667 | 1317.533333 | 102.8198 |
| | 682.6 | | | |
| | 688.1 | | | |
| Reference | 754.8 | 718.6 | 1281.4 | 100 |
| sample 1256 | | | | |
| | 707.2 | | | |
| | 693.8 | | | |
| Control | 1850 | | | |
| Control after | 1700 | | | |
| 6 hours | | | | |

It can be seen from the Tables that the relative concentrations of superoxides do not appreciably degrade while in the borosilicate bottles. Sample 397 had a decayed about 5% and sample 512 had 0% decay. Therefore, the yearly decay of product is no more than about 2.5% decay per year. This gives an estimated half-life of the superoxides at about 24 years.

The stability of any component in the composition can be measured by the amount of the particular composition which remains detectable after a certain amount of time. For example, if the superoxides measured had a decay rate of about 7% over a two year period, this would mean that the stability over the 2 year period was about 93%. In other words, after a two year period, about 93% of the original amount of superoxides, were still present and measured in the composition.

### Example 9

### Effects of RXN Beverage Intake on Endurance Performance in Mice

Animals: Six-month old male specific pathogen-free C57BL/6 mice (n=60) were purchased from Jackson Laboratory. Mice were randomly assigned to each of the four treatment groups (n=15 each). Mice were group housed (3-4/cage) and provided standard rodent chow and water ad libitum. All animal procedures were reviewed and approved by the North Carolina Research Campus IACUC.

Treatment and Design: A beverage as characterized similar to that of Example 2 and/or made by a process similar to that of Example 1, which is called ASEA in this study, or placebo (same ingredients as ASEA beverage without undergoing the processing as described in Example 1) was administered via gavage once per day for 1-week. The average body mass of all the mice at the start of the study determined the volume of ASEA used for the gavaging, but the volume did not exceed 0.3 mL. Following the 1-week treatment period (7 days) mice were euthanized and tissues harvested for further analysis of outcome measures. Mice from the endurance testing treatment groups were oriented to the treadmill in the following fashion: During the three day period preceding the maximal endurance test, mice were oriented (trained) to the treadmill for 15 min/day. Speeds for the training days were 10 m/min, 15 m/min, and 18 m/min respectively. Then, on the final day of treatment mice underwent the maximal endurance capacity test on the treadmill (Table 7). For the treadmill orientation and endurance protocols, mice were run on a multi-lane rodent treadmill (Columbus Instruments, Columbus Ohio) equipped with a shock grid at the back. When the mouse could either no longer run (as assessed by sitting on the shock grid with all 4 paws off of the belt for more than 5 seconds), the mouse was removed from the shock grid immediately and placed back into the home cage. The mice were monitored for recovery for a period of at least 20 minutes following the orientation bouts. Mice were euthanized within 30 minutes of the final endurance test.

TABLE 7
Treadmill Endurance Protocol

| Time | | |
|---|---|---|
| (min) | Speed (m/min) | Details |
| 1 | 0 | adjustment to treadmill |
| 5 | 10 | “warm up” |
| 2 | 12 | |
| 2 | 14 | |
| 2 | 16 | |
| 2 | 18 | |
| 2 | 20 | |
| 2 | 22 | Speeds between 20-24 correspond to roughly |
| | | 80% VO2max for mice |
| 2-end | 24 | Mice will stay at this speed until they reach |
| | | exhaustion (sit on shock grid for S full seconds) |

Glycogen: Post-exercise and end point liver and muscle glycogen levels were assayed using the Glycogen Assay Kit (700480, Cayman Chemical Company, Ann Arbor Mich.). Rate of muscle glycogen usage was estimated for both ASEA Run and Placebo Run groups. Example Calculation: Average muscle glycogen (Placebo Sedentary)−Average muscle glycogen (Placebo Run)/Average Placebo Run Time.

Enzyme Assays: β-hydroxyacyl-CoA dehydrogenase (β-HAD) activities were determined in whole gastrocnemius homogenates using methods previously described (Laye, 2009). Briefly, powdered frozen muscle was homogenized in buffer containing HEPES, Na pyrophosphate, Na+, EDTA, Triton, and protease and phosphatase inhibitors. CS activity was measured in homogenate incubated in buffer containing oxaloacetate and dithiobis(2-nitrobenzoic acid) (DTNB). Acetyl-CoA was added to the buffer and CS activity was determined by the appearance of reduced DTNB at a wavelength of 405 nm. 3-HAD activity was measured in homogenate incubated in buffer containing triethanolamine, EDTA, and nicotinamide adenine dinucleotide (NADH). Acetyl-CoA was added to the buffer and 3-HAD activity was determined by the disappearance of NADH at a wavelength of 340 nm. All assays were performed at 37° C.

Western Blotting: Western blotting was performed as previously described (Laye et al. 2009). The following antibodies were used: Carnitine Palmitoyltransferase-1 (CPT1) (Santa Cruz Biotechnology, Santa Cruz, Calif.), Acetyl-CoA Carboxylase (ACC), and phospho-ACC (Ser79) (Cell Signaling, Danvers, Mass.). Whole gastrocnemius homogenates were separated by SDS-PAGE, transferred to polyvinylidene fluoride membranes. Membranes were exposed to the appropriate primary and secondary antibodies and bands were visualized by chemiluminescence (Pierce SuperSignal, Fisher Scientific, Rockford, Ill.). Band density was determined using a ChemiDoc XRTS Molecular Imager and Image Lab Software (BioRad, Hercules, Calif.). Phosphorylated-ACC (Ser79) protein was normalized to total ACC protein.

Statistical Analyses: Two-way ANOVA was performed. Following a significant F-ratio, Student's t-test were performed to determine differences between treatments. Significance was established at P<0.05.

### CONCLUSIONS

When adjusted to run time, the estimated rate of muscle glycogen depletion was different between ASEA Run and Placebo Run groups. FIG. 27 graphically represents the data showing less glycogen depletion in mice that were given a composition as described herein. As can be seen, there was about a 30% decrease in the rate of muscle glycogen depletion in the ASEA group as compared to the Placebo group. A 30% decrease in the rate of muscle glycogen depletion is indicative of glycogen sparing.

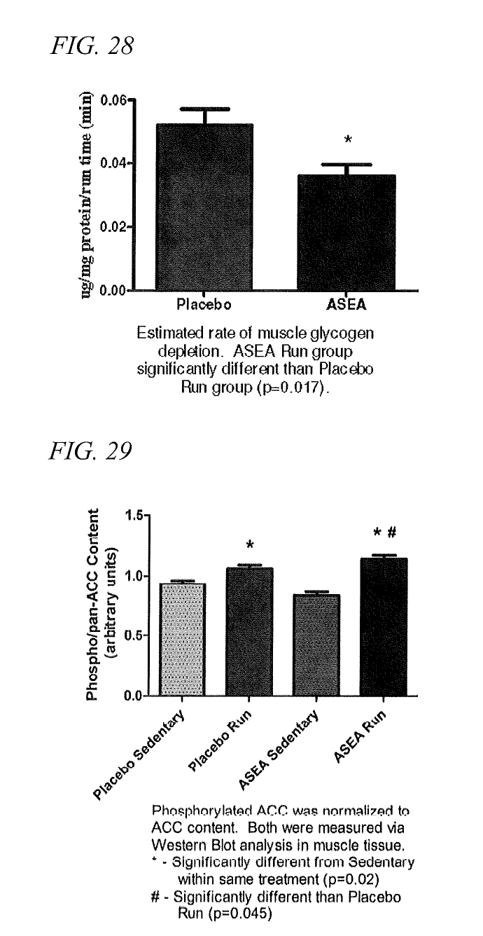

Referring to FIG. 28, skeletal muscle phosphorylated acetyl-CoA carboxylase (p-ACC) was significantly increased in ASEA Run compared to ASEA Sedentary (p=0.020) and Placebo Run groups (p=0.045). These data are consistent with a physiological scenario of increased fat burning and a sparing of glycogen.

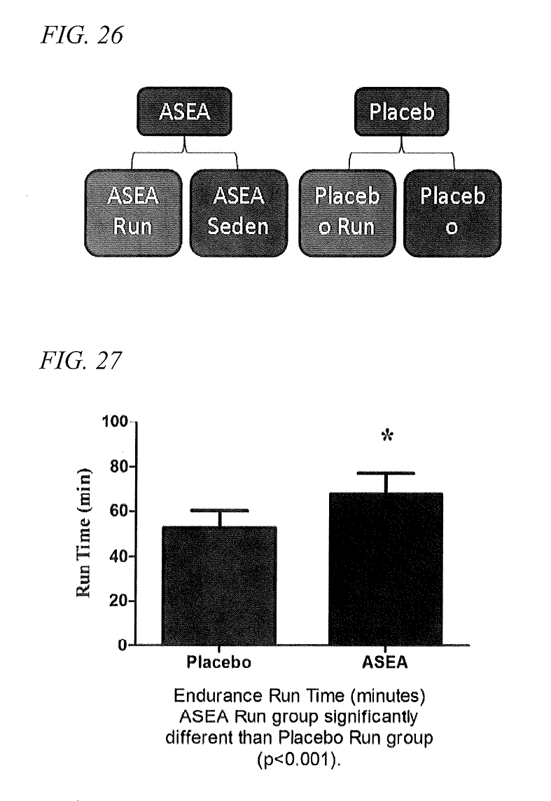

ASEA increased run time to exhaustion by 29% in mice as shown in FIG. 26, potentially through less inhibition of fatty acid oxidation via increased P-ACC, and muscle glycogen sparing (30%).

Without being bound to theory, it is believed that this 30% decrease in the rate of muscle glycogen depletion supported conditions which favor exercise as shown by an increase in run time to exhaustion by 29% in mice, potentially through less inhibition of fatty acid oxidation via increased P-ACC. Therefore, it is shown that a decrease in the rate of muscle glycogen depletion is proportional to an increase in endurance. To that end, the change in muscle glycogen depletion is a benchmark for physical endurance. The greater the decrease in muscle glycogen depletion, the greater the physical endurance. Given the beneficial impact of ASEA on the health and wellbeing of individuals who take it, this 30% decrease in muscle glycogen depletion is a benchmark itself. In one embodiment, a decrease in muscle glycogen depletion following exercise correlates with an increase in athletic endurance. In another embodiment, at least a 1% decrease in muscle glycogen depletion is indicative of an increase in athletic endurance. In another embodiment, at least a 5% decrease in muscle glycogen depletion is indicative of an increase in athletic endurance. In another embodiment, at least a 10% decrease in muscle glycogen depletion is indicative of an increase in athletic endurance. In another embodiment, at least a 15% decrease in muscle glycogen depletion is indicative of an increase in athletic endurance. In another embodiment, at least a 20% decrease in muscle glycogen depletion is indicative of an increase in athletic endurance. In another embodiment, at least a 25% decrease in muscle glycogen depletion is indicative of an increase in athletic endurance.

Without being bound to theory, it is believed that p-ACC upregulation increases fatty acid oxidation which in turn spares muscle glycogen. To this end, p-ACC upregulation is compared to an average p-ACC expression of a known population. A comparison of p-ACC upregulation to a known sample provides a benchmark for determining a change in fatty acid oxidation, muscle glycogen sparing and then therefore, an endurance predictor. In other words, the use of p-ACC as an indicator of physical or athletic endurance is one embodiment of the present invention.

The data support increased endurance capacity and altered substrate utilization in mice after one week of ASEA intake. Further research is warranted to determine if these findings are due to hormesis influences from the ASEA beverage.

The theory of hormesis involves repeated exposure to a mild physical, chemical, or biological stress resulting in increased resistance to subsequent exposures to otherwise harmful doses of the same stressors. The exposures to mild stressors are thought to induce beneficial cellular responses leading to increased whole organism resistance to the stress. Common examples of this beneficial response include, exercise, ischemic preconditioning, and caloric restriction (Mattson, 2008). ASEA may increase exercise performance through a hormesis effect, but this has not yet been established.

### Example 10

### Effect of an Immune-Supporting Supplement, ASEA, on Athletic Performance

Described is a pilot study used to measure the possible effects of an immune-supporting supplement on athletic performance as measured by a standard VO2max and Ventilatory Threshold (VT) athletic endurance test.

The objectives of the pilot study were to (1) confirm the general observation that an immune-supporting supplement has an effect on athletic performance and (2) determine the specific physiological parameters: Heart Rates (HR), volume of O2 inspired (VO2), volume of CO2 expired (VCO2), volume of expired gas (VE), Respiration Rate (RR), Respiratory Exchange Ratio (RER), Aerobic Threshold (AeT), Anaerobic Threshold (AT), VO2max and Ventilatory Threshold (VT) that are affected by oral ingestion of this supplement during both the aerobic and anaerobic phases of exercise.

The immune-supporting supplement, ASEA which in this case was made according to the process described in Example 1, contains a balanced mixture of Redox Signaling molecules that purportedly increases the efficiency of the communication channels between cells, enabling faster response of the immune system and cellular healing activities. Enzymes in the body also break down these Redox Signaling molecules into salt water and nascent oxygen. There are two proposed mechanisms involving Redox Signaling that can affect athletic performance, (1) increased efficiencies in cellular absorption or use of oxygen, prolonging aerobic metabolism, and (2) more efficient processing of lactate energy stores and tissue repair mechanisms, prolonging anaerobic metabolism.

During physical activity, the increased power requirements from muscle tissues require increased metabolism of available energy stores. Sustainable aerobic metabolism of sugars can supply this energy demand as long as there is an adequate supply of oxygen and sugars in the blood. As energy demands exceed the ability of the respiratory and cardiovascular system to deliver sufficient oxygen to the muscle tissue, methods involving the anaerobic metabolism of carbohydrates, creatines, pyruvates, etc. start to become prevalent.

Anaerobic metabolism supplies the excessive demand for energy but is accompanied by the production of CO2 and lactates. Prolonged or excessive anaerobic metabolism depletes the available energy stores faster than they can be renewed; the buildup of CO2 and lactates can also interfere with aerobic metabolism and thus, when the energy stores are spent, exhaustion will result.

Because anaerobic metabolism is marked by an excess in CO2 and lactate production, it can be monitored by measuring the excess CO2 exhaled during exercise or the buildup of lactates in the blood. The Ventilatory Threshold (VT) is the point where the excess CO2 is first detected in the expired breath; it is related to the point at which anaerobic metabolism is starting to become prevalent.

In this pilot study, VT was determined graphically from the VCO2 vs. VO2 graph. VCO2 is the volume of CO2 expired per minute and VO2 is the volume of 02 inspired per minute. VO2max is simply the maximum volume of 02 inspired per minute possible for any given individual. VO2max is measured in mL/kg/min (milliliters of 02 per kilogram of body weight per minute). VO2max is measured at the peak of the VO2 curve. The Aerobic Threshold (AeT) was determined by the software and indicates when fat-burning metabolic activities start to be dominated by aerobic metabolism. The Anaerobic Threshold (AT) was also software-determined and marks the point where the anaerobic metabolism starts to completely dominate.

Recruitment Methods: A standard VO2max test was run on 18 athletes who responded to recruitment flyers posted in athletic clubs and to invitations extended to a local competitive Triathlon team. The participants were selected based on answers from qualification questionnaire which affirmed that they:

1. Perform a rigorous physical workout at least five hours per week on average.

2. Have no medical conditions that might prevent participation

3. Agree to follow diet and hydration instructions.

4. Will perform only normal daily routines during the study.

5. Have no history of heart problems in the family.

The final selections were athletes of a caliber much higher that the expectations reflected in the recruitment flyers, a majority being athletes involved in regular athletic competitions. All of the participants had never taken the supplement prior to the study.

The participants did not receive any monetary compensation, but did receive a case of product and results from the VO2max tests.

The VO2max testing was done at an athletic club by accredited professionals holding degrees in exercise physiology and with more than 10 years daily experience in administering VO2 tests. The participants were given a choice of performing the test on either a treadmill or a stationary cycle. A CardioCoach® metabolic cart measured heart rate (HR), inspired and expired gases (VO2, VCO2, VE) and recorded weight, height, age, and body mass indexes (BMI). Power settings on the treadmill or cycle were recorded every minute.

Each participant was scheduled to take two VO2max tests, (1) a baseline test and (2) a final test. The baseline test was performed before any supplement ingestion. The participants drank 4 oz. of the supplement per day between the baseline test and the final test (7 to 10 days later) and drank 8 oz. of the supplement ten minutes before starting the final test. For the baseline test, the power settings on the cycle or treadmill were determined by the test administrator. The power settings for the final test were matched exactly to the power settings of the baseline test for each participant. Participants were encouraged to strictly maintain their regular diet and exercise routine and to come to each test well hydrated (at least 8 oz. of water in the last 2 hours before each test).

Each participant was fitted with a breathing mask and heart monitor. Each VO2max test consisted of a 10 min. warm up period where participants walked or cycled at a low power setting determined by the administrator. This was followed by a ramp up period, where the administrators increased the power settings every minute, according to their evaluation of the physical condition of the participant, and termination when the administrators started seeing the indications of a maximum VO2 reading when RER (VCO2/VO2)>1.0 or at the administrator's discretion. The administrators had ample experience in obtaining consistent VO2max results on this equipment, estimated at about 6% test to test variation over the last 5 years.

The raw data (HR, VO2, VCO2, VE, Power Settings) were collected from the CARDIOCOACH® software for analysis. Data points were automatically averaged over 15 to 25 second breath intervals by the software, VO2max is also determined by the software with an averaged VO2 peak method. VT was determined graphically from the slope of the VCO2 vs. VO2 graph.

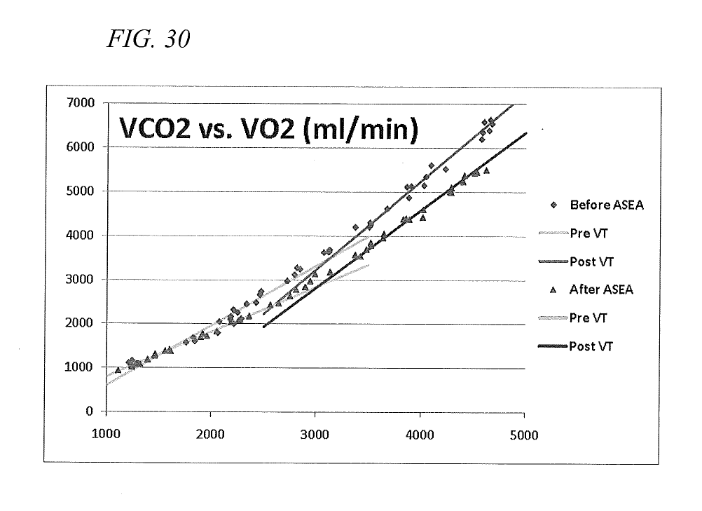

Linear regression methods were used to determine the slope, change in VCO2 over change in VO2. In theory, when aerobic metabolism switches to anaerobic metabolism, the volume of CO2 expelled (VCO2) is increased in proportion to the Volume of 02 inhaled (VO2). This is reflected as an increase of slope on the VCO2 vs. VO2 graph, seen as a clear kink on the graph around the VT point. Linear regression was used to determine the slope both before VT and after. Slopes were determined by linear regression on the linear region of data points before and after VT point, excluding points surrounding the VT and near VO2max. The intersection of the before and after lines was used to determine the reported VT point (FIG. 30).

Methods for determining the VT point on any individual participant were kept consistent from the baseline test to the final test. Average HR was averaged over the linear range of HR increase during the power ramp, excluding points a few minutes into the beginning and before the end of the data set. In every case, the same data analysis methods were used for the final test as were used for the baseline test for each participant.

Compliance to protocol was very high by both participants and administrators, based on answers to compliance questions. One data set was discarded for low VCO2 values, probably due to a loose mask. The ventilatory data for this one participant was rejected, leaving 17 valid data ventilatory data sets. The Heart Rates (HR), however, were compared for all 18 participants. The results are show below:

| Total | | | Average | | | |
|---|---|---|---|---|---|---|
| Partici- | Average | Male/ | Weight | Cycle/ | Average | Data Sets |
| pants | Age | Female | (Kg) | Treadmill | BMI | Selected |
| 18 | 41 ± 9 | 16/2 | 76 ± 11 | 7/11 | 24.4 ± 3.4 | 17 |

The average VO2max reading over all participants (N=17) was measured at the relatively high value of 62.5 mL/kg/min, indicative of the quality of athletes in the sample. Only four participants had VO2max readings below 55 mL/kg/min; these four were not involved in competitive training programs.

The data shows that two significant changes in physiological parameters could be attributed to ingestion of the composition, as determined by a statistical paired t-test analysis. The average time taken to arrive at VO2max was increased by 10% with very high confidence (P=0.006) and the average time taken to arrive at Ventilatory Threshold (VT) was increased by 12% with a marginal level of confidence (P=0.08).

Given that the power ramp-up-points between the baseline and final test for each participant were identical, an increase in the amount of time to obtain VO2max and VT on the final test also indicates a higher average power outputs at such thresholds. Calibrated power output measurements were not available. However, the test administrator for the final test, upon reaching the maximum power recorded for the baseline test, regularly surpassed this maximum power before the participant reached VO2max on the final test.

All other physiological parameters (VO2max, VT, AeT, AT, Start HR, HR at AeT, HR at AT, HR at VO2max, and overall average HR) were not significantly changed by supplement ingestion. The high level of consistency between the baseline and final test for these parameters, however, supports the repeatability of the tests. The test to test repeatability has an estimated standard deviation of less than 5% for all parameters (as shown in the following Table below).

| Averages | | | | | |
|---|---|---|---|---|---|
| (N = 17) | Baseline | Final | Change | % Change | P-Value |
| VO₂ₘₐₓ | 62.5 | 63.6 | +1.1 | +2% | — |
| (mL/kg/min) | | | | | |
| VT | 36.4 | 38.7 | +2.3 | +6% | 0.34 |
| (mL/kg/min) | | | | | |
| Aerobic | 43.6 | 43.8 | +0.2 | +0% | — |
| Thresh. (AeT) | | | | | |
| Anaerobic | 55.5 | 56.5 | +1.0 | +2% | — |
| Thresh. (AT) | | | | | |
| Pre VT Slope | 1.030 | 1.030 | 0.0 | 0% | — |
| of VCO₂/VO₂ | | | | | |
| Post VT | 1.997 | 1.944 | −0.053 | −2.7% | — |
| slope of | | | | | |
| VCO₂/VO₂ | | | | | |
| Start Heart | 87.4 | 85.9 | −1.5 | −1% | — |
| Rate (bpm) | | | | | |
| Heart Rate at | 147 | 145 | −2 | −2% | — |
| AeT | | | | | |
| Heart Rate at | 165 | 165 | 0 | 0% | — |
| AT | | | | | |
| Heart Rate at | 174 | 175 | +1 | +1% | — |
| VO₂ₘₐₓ | | | | | |
| Heart Rate | 137 | 134 | −3 | −2% | — |
| Overall | | | | | |
| Time to VT | 306 | 344 | 38 | +12% | 0.08 |
| (secs) | | | | | |
| Time to | 639 | 703 | 64 | +10% | 0.006 |
| VO₂ₘₐₓ(s) | | | | | |

Of the 17 participants in the study, 70% of them experienced a significant increase in time to VO2max, 18% of the participants showing more than a 25% increase, 41% showing more than a 10% increase, 18% of the participants exhibiting no significant change and 12% showing a mild decrease (under 10%).

There was a moderate but significant correlation between the increases in “time to VO2max” and “time to VT” (correlation coefficient 0.35), meaning that an increase in time to reach VO2max was moderately but not always proportional to the increase in the time it took to get to VT. There is a strong correlation between increase in time to VO2max and decrease in the average overall heart rate (correlation coefficient −0.67), meaning that an increase in time to VO2max would most often be accompanied by a decrease in average overall heart rate.

Ingestion of the test supplement, ASEA, for 7-10 days prior to and immediately before a VO2max test, was shown to significantly increase the time it took for 70% of the participants to reach VO2max under equivalent carefully regulated power ramp-up conditions. Time to VT likewise was significantly extended.

The extension of time to reach VT, under similar increasing demands for energy, is a direct indication that the aerobic phase of metabolism is being extended and/or the anaerobic phases somehow are being delayed as the demand for energy increases.

The lack of any other changes in the physiological parameters (VO2max, VT, AeT, AT and associated heart rates) suggests that cardiovascular capacity, lung capacity and blood oxygen capacity and regulation were not affected. This assumption is reasonable, given that the short duration of this study excluded the possibility of training effects.

One feasible explanation for the results lies in the enhancement of aerobic efficiencies, meaning that more aerobic energy can be extracted at the same physiological state, or that the clearance of lactates or CO2 becomes more efficient, again allowing greater aerobic efficiency. Note that “time to AeT” and “time to AT” were not compiled in this study, however changes in these parameters would be expected and might offer clues to determine the underlying mechanisms.

The results of this pilot test indicate that there is a strong case for athletic performance enhancement and further investigation is warranted. A placebo-based double-blind test, measuring the more subtle effects in ventilation and heart rates along with increases in blood lactate levels during a controlled, calibrated power ramp would provide defensible evidence for this effect and better support for some specific underlying mechanisms of action.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

It is contemplated that numerical values, as well as other values that are recited herein are modified by the term “about”, whether expressly stated or inherently derived by the discussion of the present disclosure. As used herein, the term “about” defines the numerical boundaries of the modified values so as to include, but not be limited to, tolerances and values up to, and including the numerical value so modified. That is, numerical values can include the actual value that is expressly stated, as well as other values that are, or can be, the decimal, fractional, or other multiple of the actual value indicated, and/or described in the disclosure.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A method of benchmarking wellness of a subject comprising measuring the rate of muscle glycogen depletion in the subject after exercise and comparing said rate of muscle glycogen depletion to the rate of muscle glycogen depletion of a known standard.

2. The method of claim 1, wherein the known standard is the post-exercise average rate of muscle glycogen depletion for a known or given population.

3. The method of claim 2, wherein the difference in the rate of muscle glycogen depletion between the subject and the known standard is measured as a percentage of the known standard.

4. The method of claim 3, wherein there is a decrease in the rate of muscle glycogen depletion in the subject compared to the known standard, and wherein the decrease in the rate of muscle glycogen depletion is from 1-35%.

5. The method of claim 4, further comprising administering to the subject a composition comprising at least one RXN wherein the RXN is selected from the group consisting of reduced species (RS) and reactive oxygen species (ROS).

6. The method of claim 5, wherein the composition comprising at least one RXN is made by electrolyzing a homogenous and well mixed solution of saline and water.

7. The method of claim 6, wherein the temperature, flow and electrical current are adjusted during the process of electrolyzing.

8. The method of claim 7, wherein the temperature at the time of electrolyzing is between 30-100° F.

9. The method of claim 7, wherein the voltage drops to zero at least once per second during the process of electrolyzing and further wherein the voltage remains positive during the process of electrolyzing.

10. The method of claim 5, wherein the composition comprises at least one ROS and the at least one ROS includes a superoxide and the superoxide is *O₂⁻.

11. The method of claim 10, wherein the presence of the superoxide is detectable for at least 10 years.

12. The method of claim 5, wherein the at least one reduced species (RS) includes HOCl, NaClO, O₂, H₂, H⁺, ClO, Cl₂, H₂O₂ or mixtures thereof and the at least one reactive oxygen species (ROS) includes O₂⁻, HO₂, Cl⁻, H⁻, *OCl, O₃, *O₂⁻, OH⁻ or mixtures thereof.

13. The method of claim 5, wherein the composition comprises hypochlorous acid or a salt thereof.

14. The method of claim 5, wherein at least 60% of the at least one reactive oxygen species (ROS) is present in the composition after 1 year.

15. The method of claim 5, wherein at least 98% of the at least one reactive oxygen species (ROS) is present in the composition after 1 year.

16. The method of claim 5, wherein at least 65% of the at least one reactive oxygen species (ROS) is present in the composition after 10 years.

17. The method of claim 5, wherein at least 100% of the at least one reactive oxygen species (ROS) is present in the composition after 10 years.

18. The method of claim 5, wherein the at least one reactive oxygen species (ROS) has a half-life of about 24 years.

19. The method of claim 5, wherein the at least one reactive oxygen species (ROS) has a half-life of greater than about 24 years.

20. The method according to any one of claims 14-19, wherein the at least one reactive oxygen species (ROS) is hypochlorous acid or a salt thereof.

21. A method of predicting physical or athletic endurance comprising testing the p-ACC of an individual and comparing the level of p-ACC expression to the average p-ACC of a known population.
